SEQUENCE LISTING (139 sequences)

SEQ ID NO: 1 - DNA, 23 bp, Artificial Sequence
  Oligonucleotide
gctcgcttcggcagcacatatac

SEQ ID NO: 2 - DNA, 23 bp, Artificial Sequence
  Oligonucleotide
gagacgtgctacttccatttgtc

SEQ ID NO: 3 - DNA, 20 bp, Artificial Sequence
  Oligonucleotide
gagggcctatttcccatgat

SEQ ID NO: 4 - DNA, 97 bp, Artificial Sequence
  Oligonucleotide
tgctgttgacagtgagcgcgcagatgaggagaagaggatttagtgaagccacagatgtaaatcctcttctcctcatctgcatgcctactgcctcgga

SEQ ID NO: 5 - DNA, 97 bp, Artificial Sequence
  Oligonucleotide
tgctgttgacagtgagcgcgcccatcctcagggaccatattagtgaagccacagatgtaatatggtccctgaggatgggcttgcctactgcctcgga

SEQ ID NO: 6 - DNA, 97 bp, Artificial Sequence
  Oligonucleotide
tgctgttgacagtgagcgcggagcgattcgcaaacatgaatagtgaagccacagatgtattcatgtttgcgaatcgctccttgcctactgcctcgga

SEQ ID NO: 7 - DNA, 97 bp, Artificial Sequence
  Oligonucleotide
tgctgttgacagtgagcgcggacatttaattctgagattatagtgaagccacagatgtataatctcagaattaaatgtccttgcctactgcctcgga

SEQ ID NO: 8 - DNA, 28 bp, Artificial Sequence
  Oligonucleotide
gttgtagatatgttgtggatttgggttg

SEQ ID NO: 9 - DNA, 31 bp, Artificial Sequence
  Oligonucleotide
ctaaacaaatctataaaccaaaatccctctc

SEQ ID NO: 10 - DNA, 27 bp, Artificial Sequence
  Oligonucleotide
gggttgttttgtttttggtaagttttg

SEQ ID NO: 11 - DNA, 24 bp, Artificial Sequence
  Oligonucleotide
ccaaaatccctctccaaccatact

SEQ ID NO: 12 - DNA, 27 bp, Artificial Sequence
  Oligonucleotide
ggagagggattttggtttatagatttg

SEQ ID NO: 13 - DNA, 27 bp, Artificial Sequence
  Oligonucleotide
ccatccacaatttaacaactcaattcc

SEQ ID NO: 14 - DNA, 26 bp, Artificial Sequence
  Oligonucleotide
aaatatccaccaattcaaccatccac

SEQ ID NO: 15 - DNA, 24 bp, Artificial Sequence
  Oligonucleotide
gtatggttggagagggattttggt

SEQ ID NO: 16 - DNA, 26 bp, Artificial Sequence
  Oligonucleotide
gaaaagaagtagaaatagaagttgag

SEQ ID NO: 17 - DNA, 26 bp, Artificial Sequence
  Oligonucleotide
ctacatcccaactataactttactac

SEQ ID NO: 18 - DNA, 26 bp, Artificial Sequence
  Oligonucleotide
gtttggtttatagttatagagtagag

SEQ ID NO: 19 - DNA, 30 bp, Artificial Sequence
  Oligonucleotide
cactaaaaaacatcattacttacactaacc

SEQ ID NO: 20 - DNA, 26 bp, Artificial Sequence
  Oligonucleotide
gtgaggttaggggtgggggaagttta

SEQ ID NO: 21 - DNA, 26 bp, Artificial Sequence
  Oligonucleotide
ctcttaactctctatatctactcctc

SEQ ID NO: 22 - DNA, 30 bp, Artificial Sequence
  Oligonucleotide
gttttatttttgtttttaatttgggttagg

SEQ ID NO: 23 - DNA, 29 bp, Artificial Sequence
  Oligonucleotide
aaatacctcaacaaatataaacctaaaaa

SEQ ID NO: 24 - DNA, 24 bp, Artificial Sequence
  Oligonucleotide
agggagggggttgttaggattttg

SEQ ID NO: 25 - DNA, 28 bp, Artificial Sequence
  Oligonucleotide
gttgttaggattttgtgatggtgagttg

SEQ ID NO: 26 - DNA, 27 bp, Artificial Sequence
  Oligonucleotide
taacaaccaccctctctcctttcactc

SEQ ID NO: 27 - DNA, 29 bp, Artificial Sequence
  Oligonucleotide
caccccaaataaaaaacccattcacttac

SEQ ID NO: 28 - DNA, 37 bp, Artificial Sequence
  Oligonucleotide
ggaagttatttagtatttttattgttttgtttatgtg

SEQ ID NO: 29 - DNA, 31 bp, Artificial Sequence
  Oligonucleotide
gttgtttttttgttgtgtgggatattagata

SEQ ID NO: 30 - DNA, 28 bp, Artificial Sequence
  Oligonucleotide
caacaactaacttactatcactttccta

SEQ ID NO: 31 - DNA, 28 bp, Artificial Sequence
  Oligonucleotide
tccaaatatcaaaaaacctacctaccta

SEQ ID NO: 32 - DNA, 22 bp, Artificial Sequence
  Oligonucleotide
ttaggaaaggtaaggggtagat

SEQ ID NO: 33 - DNA, 29 bp, Artificial Sequence
  Oligonucleotide
caatcatttactccaaataaaactccatc

SEQ ID NO: 34 - DNA, 25 bp, Artificial Sequence
  Oligonucleotide
agttagggattgtttttagtttagg

SEQ ID NO: 35 - DNA, 24 bp, Artificial Sequence
  Oligonucleotide
cacaactccccraaactcccattc

SEQ ID NO: 36 - DNA, 26 bp, Artificial Sequence
  Oligonucleotide
atttgttatttagtttgggttgggat

SEQ ID NO: 37 - DNA, 27 bp, Artificial Sequence
  Oligonucleotide
ctacatctctaactacaactaaaacac

SEQ ID NO: 38 - DNA, 28 bp, Artificial Sequence
  Oligonucleotide
gaaagtatttgtttagtttttggttttg

SEQ ID NO: 39 - DNA, 28 bp, Artificial Sequence
  Oligonucleotide
caaattaaacaacaccattcttataacc

SEQ ID NO: 40 - DNA, 25 bp, Artificial Sequence
  Oligonucleotide
gttttgttttttaaggggtgttgat

SEQ ID NO: 41 - DNA, 23 bp, Artificial Sequence
  Oligonucleotide
ttataacacaacctcaaatccac

SEQ ID NO: 42 - DNA, 21 bp, Artificial Sequence
  Oligonucleotide
cagcccagagtaactcacttc

SEQ ID NO: 43 - DNA, 21 bp, Artificial Sequence
  Oligonucleotide
catacccacaggcagtctaga

SEQ ID NO: 44 - DNA, 21 bp, Artificial Sequence
  Oligonucleotide
gaagtagaaacagaagctgag

SEQ ID NO: 45 - DNA, 21 bp, Artificial Sequence
  Oligonucleotide
ttgctacatcccaactgtaac

SEQ ID NO: 46 - DNA, 21 bp, Artificial Sequence
  Oligonucleotide
ggtctacagccacagagcaga

SEQ ID NO: 47 - DNA, 22 bp, Artificial Sequence
  Oligonucleotide
tcttctgtcactagagggcatc

SEQ ID NO: 48 - DNA, 20 bp, Artificial Sequence
  Oligonucleotide
gccactggttataaagtcca

SEQ ID NO: 49 - DNA, 20 bp, Artificial Sequence
  Oligonucleotide
aaagctgtttccctcccgac

SEQ ID NO: 50 - DNA, 21 bp, Artificial Sequence
  Oligonucleotide
gctgctaggaccttgtgatgg

SEQ ID NO: 51 - DNA, 20 bp, Artificial Sequence
  Oligonucleotide
caccccagatgagaggccca

SEQ ID NO: 52 - DNA, 21 bp, Artificial Sequence
  Oligonucleotide
gctgtttctttgttgtgtggg

SEQ ID NO: 53 - DNA, 21 bp, Artificial Sequence
  Oligonucleotide
tccagatgtcaggggacctgc

SEQ ID NO: 54 - DNA, 20 bp, Artificial Sequence
  Oligonucleotide
caggccggcgagtttgccgg

SEQ ID NO: 55 - DNA, 20 bp, Artificial Sequence
  Oligonucleotide
tgcgggtggcccggaagagc

SEQ ID NO: 56 - DNA, 20 bp, Artificial Sequence
  Oligonucleotide
gatcgagaagaggaagctgc

SEQ ID NO: 57 - DNA, 20 bp, Artificial Sequence
  Oligonucleotide
tctgggtcggggtaacttcc

SEQ ID NO: 58 - DNA, 20 bp, Artificial Sequence
  Oligonucleotide
cgccccgagccttgatttag

SEQ ID NO: 59 - DNA, 20 bp, Artificial Sequence
  Oligonucleotide
actcaggcgtcgccgtcaga

SEQ ID NO: 60 - DNA, 20 bp, Artificial Sequence
  Oligonucleotide
ctgattggctgcgcgcgggg

SEQ ID NO: 61 - DNA, 20 bp, Artificial Sequence
  Oligonucleotide
gcagtgccgggccccgtccg

SEQ ID NO: 62 - DNA, 19 bp, Artificial Sequence
  Oligonucleotide
gccgcctgtgcagtggtga

SEQ ID NO: 63 - DNA, 22 bp, Artificial Sequence
  Oligonucleotide
ctgtgtgtgggcagcctgcggc

SEQ ID NO: 64 - DNA, 22 bp, Artificial Sequence
  Oligonucleotide
ggctatagatcaccttcatgta

SEQ ID NO: 65 - DNA, 21 bp, Artificial Sequence
  Oligonucleotide
gcagttaactcagggaccaag

SEQ ID NO: 66 - DNA, 20 bp, Artificial Sequence
  Oligonucleotide
gcgttgccatagcatgaact

SEQ ID NO: 67 - DNA, 20 bp, Artificial Sequence
  Oligonucleotide
gagttaggggaccatagtca

SEQ ID NO: 68 - DNA, 20 bp, Artificial Sequence
  Oligonucleotide
tatggcgaggaaaactgaaa

SEQ ID NO: 69 - DNA, 21 bp, Artificial Sequence
  Oligonucleotide
ttcacgtcctaaagtgtgtat

SEQ ID NO: 70 - DNA, 22 bp, Artificial Sequence
  Oligonucleotide
ggcaaaggttcaggtgaacaat

SEQ ID NO: 71 - DNA, 21 bp, Artificial Sequence
  Oligonucleotide
ggatgagtcctgcattcggag

SEQ ID NO: 72 - DNA, 21 bp, Artificial Sequence
  Oligonucleotide
cactcctctagcacctcacac

SEQ ID NO: 73 - DNA, 20 bp, Artificial Sequence
  Oligonucleotide
ggtgatggaggtgtgaggtg

SEQ ID NO: 74 - DNA, 20 bp, Artificial Sequence
  Oligonucleotide
tcggccaacttgcaaaagaa

SEQ ID NO: 75 - DNA, 19 bp, Artificial Sequence
  Oligonucleotide
gggactggcaaacaggaag

SEQ ID NO: 76 - DNA, 20 bp, Artificial Sequence
  Oligonucleotide
accttggtcgagtcgcttcc

SEQ ID NO: 77 - DNA, 20 bp, Artificial Sequence
  Oligonucleotide
aactcgtcgccttccaggtc

SEQ ID NO: 78 - DNA, 20 bp, Artificial Sequence
  Oligonucleotide
cacgggggagaagccttatg

SEQ ID NO: 79 - DNA, 20 bp, Artificial Sequence
  Oligonucleotide
cgggtgaatgttttcccaca

SEQ ID NO: 80 - DNA, 20 bp, Artificial Sequence
  Oligonucleotide
gaaccatcaccgtgcgagac

SEQ ID NO: 81 - DNA, 20 bp, Artificial Sequence
  Oligonucleotide
ccagtgggctcatgtccttg

SEQ ID NO: 82 - DNA, 20 bp, Artificial Sequence
  Oligonucleotide
ccaccccataccaggaccat

SEQ ID NO: 83 - DNA, 20 bp, Artificial Sequence
  Oligonucleotide
ctgctgggctcatcctcaga

SEQ ID NO: 84 - DNA, 21 bp, Artificial Sequence
  Oligonucleotide
cgagaggggaagtgtcgactg

SEQ ID NO: 85 - DNA, 20 bp, Artificial Sequence
  Oligonucleotide
gcctccctccaggttcttgc

SEQ ID NO: 86 - DNA, 20 bp, Artificial Sequence
  Oligonucleotide
gtagcattcagcggggctct

SEQ ID NO: 87 - DNA, 20 bp, Artificial Sequence
  Oligonucleotide
gggttgcatccaccacaaaa

SEQ ID NO: 88 - DNA, 21 bp, Artificial Sequence
  Oligonucleotide
ggctggatctggagactgacc

SEQ ID NO: 89 - DNA, 20 bp, Artificial Sequence
  Oligonucleotide
ctgcaccttcagcacctcag

SEQ ID NO: 90 - DNA, 21 bp, Artificial Sequence
  Oligonucleotide
gatgcacactctgcgatgaag

SEQ ID NO: 91 - DNA, 22 bp, Artificial Sequence
  Oligonucleotide
cagtgttcacagccaggagaat

SEQ ID NO: 92 - DNA, 20 bp, Artificial Sequence
  Oligonucleotide
gcagtccagggagctagacg

SEQ ID NO: 93 - DNA, 22 bp, Artificial Sequence
  Oligonucleotide
gagctgatcatactgtgctaag

SEQ ID NO: 94 - DNA, 19 bp, Artificial Sequence
  Oligonucleotide
gagcagtggttccgagtga

SEQ ID NO: 95 - DNA, 20 bp, Artificial Sequence
  Oligonucleotide
ggtcttcgttcagggcgatg

SEQ ID NO: 96 - DNA, 20 bp, Artificial Sequence
  Oligonucleotide
cctggacctgttgctgatgg

SEQ ID NO: 97 - DNA, 20 bp, Artificial Sequence
  Oligonucleotide
ctggagcacagctgcagtca

SEQ ID NO: 98 - DNA, 21 bp, Artificial Sequence
  Oligonucleotide
ctcatctgcctgaaagcacac

SEQ ID NO: 99 - DNA, 21 bp, Artificial Sequence
  Oligonucleotide
gggcaaagaggtacatcgaag

SEQ ID NO: 100 - DNA, 20 bp, Artificial Sequence
  Oligonucleotide
acagagtcctccccgtctgc

SEQ ID NO: 101 - DNA, 20 bp, Artificial Sequence
  Oligonucleotide
gcatgtttgggtgccatcat

SEQ ID NO: 102 - DNA, 20 bp, Artificial Sequence
  Oligonucleotide
aagaggattcccccggacat

SEQ ID NO: 103 - DNA, 20 bp, Artificial Sequence
  Oligonucleotide
acacatcgctgcctttggaa

SEQ ID NO: 104 - DNA, 20 bp, Artificial Sequence
  Oligonucleotide
acaagcctgtcggggtgaat

SEQ ID NO: 105 - DNA, 20 bp, Artificial Sequence
  Oligonucleotide
actgtgggggtccacatcct

SEQ ID NO: 106 - DNA, 22 bp, Artificial Sequence
  Oligonucleotide
ggagccctgaagccatattgag

SEQ ID NO: 107 - DNA, 21 bp, Artificial Sequence
  Oligonucleotide
ggcctctaaaggtgcaagtct

SEQ ID NO: 108 - DNA, 21 bp, Artificial Sequence
  Oligonucleotide
ccccgaacagacagaagtggt

SEQ ID NO: 109 - DNA, 21 bp, Artificial Sequence
  Oligonucleotide
cttgcatcagcctcacaagtc

SEQ ID NO: 110 - DNA, 20 bp, Artificial Sequence
  Oligonucleotide
gcaactacgacaatctcctg

SEQ ID NO: 111 - DNA, 20 bp, Artificial Sequence
  Oligonucleotide
cctttcgcttatccactgga

SEQ ID NO: 112 - DNA, 20 bp, Artificial Sequence
  Oligonucleotide
gcagccacagtttcagttgc

SEQ ID NO: 113 - DNA, 20 bp, Artificial Sequence
  Oligonucleotide
ctctggctctggctcaggat

SEQ ID NO: 114 - DNA, 20 bp, Artificial Sequence
  Oligonucleotide
tccagcagaccttccagcag

SEQ ID NO: 115 - DNA, 20 bp, Artificial Sequence
  Oligonucleotide
cctttagggccgatgaccag

SEQ ID NO: 116 - DNA, 20 bp, Artificial Sequence
  Oligonucleotide
tggggctattgcagaggtga

SEQ ID NO: 117 - DNA, 20 bp, Artificial Sequence
  Oligonucleotide
ctgctcagcctcccattcct

SEQ ID NO: 118 - DNA, 20 bp, Artificial Sequence
  Oligonucleotide
catccatggggctacagtga

SEQ ID NO: 119 - DNA, 20 bp, Artificial Sequence
  Oligonucleotide
tggcatggtgagttttcagg

SEQ ID NO: 120 - DNA, 20 bp, Artificial Sequence
  Oligonucleotide
ctcgtcgtggcctcagagaa

SEQ ID NO: 121 - DNA, 20 bp, Artificial Sequence
  Oligonucleotide
tgtgacggccttggatcact

SEQ ID NO: 122 - DNA, 20 bp, Artificial Sequence
  Oligonucleotide
cttccccagggacaaactgg

SEQ ID NO: 123 - DNA, 20 bp, Artificial Sequence
  Oligonucleotide
cggatctgcagcgatgtacc

SEQ ID NO: 124 - DNA, 20 bp, Artificial Sequence
  Oligonucleotide
tggagaagcaggagccagtg

SEQ ID NO: 125 - DNA, 20 bp, Artificial Sequence
  Oligonucleotide
ttggtaagccctgccctcat

SEQ ID NO: 126 - DNA, 20 bp, Artificial Sequence
  Oligonucleotide
ggtgaagagggagcgaagga

SEQ ID NO: 127 - DNA, 20 bp, Artificial Sequence
  Oligonucleotide
ctgtgggcaccagagattgg

SEQ ID NO: 128 - DNA, 21 bp, Artificial Sequence
  Oligonucleotide
gctggagatggctatggacac

SEQ ID NO: 129 - DNA, 20 bp, Artificial Sequence
  Oligonucleotide
gcaaccgtcagagctgccac

SEQ ID NO: 130 - DNA, 20 bp, Artificial Sequence
  Oligonucleotide
ggagaaattgcgggatgctc

SEQ ID NO: 131 - DNA, 20 bp, Artificial Sequence
  Oligonucleotide
gcccaacgacagttcaccag

SEQ ID NO: 132 - DNA, 20 bp, Artificial Sequence
  Oligonucleotide
tttcgtctggccaacagcat

SEQ ID NO: 133 - DNA, 20 bp, Artificial Sequence
  Oligonucleotide
ctgaaggatggggagggatg

SEQ ID NO: 134 - DNA, 20 bp, Artificial Sequence
  Oligonucleotide
agcttggaaggcccagtcag

SEQ ID NO: 135 - DNA, 20 bp, Artificial Sequence
  Oligonucleotide
gccttggtgctccaacacac

SEQ ID NO: 136 - DNA, 20 bp, Artificial Sequence
  Oligonucleotide
ggatgagtggaagaagctgg

SEQ ID NO: 137 - DNA, 20 bp, Artificial Sequence
  Oligonucleotide
ctccttgttgcaacacggag

SEQ ID NO: 138 - DNA, 45 bp, Artificial Sequence
  Oligonucleotide
gagaaagatgccggcggatttcaggagcagataaggaaaagattg

SEQ ID NO: 139 - DNA, 42 bp, Artificial Sequence
  Oligonucleotide
ctcctgaaatccgccggcatctttctccacctcccagggatc

(19) United States
(12) Patent Application Publication
(10) Pub. No.: US 2010/0137411 A1
(43) Pub. Date: Jun. 3, 2010

(54) RAS-MEDIATED EPIGENETIC SILENCING EFFECTORS AND USES THEREOF

(75) Inventors: Michael R. Green, Boylston, MA (US); Claude Gazin, Paris (FR); Narendra Wajapeyee, New Haven, CT (US)

Correspondence Address: WOLF GREENFIELD & SACKS, P.C., 600 ATLANTIC AVENUE, BOSTON, MA, 02210-2206, US

(73) Assignee: University of Massachusetts, Boston, MA (US)

(21) Appl. No.: 12/670,586

(22) PCT Filed: Aug. 5, 2008
(86) PCT No.: PCT/US08/09407

§ 371 Date: Jan. 26, 2010

Related U.S. Application Data
(60) Provisional application No. 60/962,047, filed on Jul. 26, 2007.

(51) Int. Cl. A61K 31/7088; C12N 5/071; C12Q 1/68; A61P 35/00

(52) U.S. Cl. 514/44 A; 435/375; 435/6

(57) ABSTRACT

The invention relates to methods for inhibiting gene silencing, methods for inhibiting cell proliferation, methods for inhibiting Ras mediated tumor growth, methods for screening for regulators of FAS expression, and methods for identifying inhibitors of Ras mediated tumor growth.

### RELATED APPLICATIONS

This application claims the benefit under 35 U.S.C. §119(e) of the filing date of U.S. Provisional Application U.S. Ser. No. 60/962,047 (Attorney Docket No.: U0120.70022US00) filed Jul. 26, 2007. The entire teachings of the referenced provisional application is expressly incorporated herein by reference.

### GOVERNMENT FUNDING

This invention was made with Government support from the National Institutes of Health under Grant No. 5-R01-GM033977-23. The Government has certain rights in the invention.

### FIELD OF THE INVENTION

The invention relates to methods for inhibiting gene silencing, methods for inhibiting cell proliferation, methods for inhibiting Ras mediated tumor growth, methods for screening for regulators of FAS expression, and methods for identifying inhibitors of Ras mediated tumor growth.

### BACKGROUND OF INVENTION

The conversion of a normal cell to a cancer cell involves a continuum of genetic and biochemical events that typically result in the activation of oncogenes and inactivation of tumour suppressors and pro-apoptotic genes (Hanahan, D. & Weinberg, R. A. Cell 100, 57-70, 2000). In many instances, inactivation of genes critical for cancer development occurs by epigenetic silencing that often involves hypermethylation of CpG-rich promoter regions (Baylin, S. B. Nat. Clin. Pract. Oncol. 2 Suppl 1, S4-11 (2005).Esteller, M. Br. J. Cancer 94, 179-183 (2006)). A long standing question has been whether this epigenetic gene silencing occurs by random acquisition of epigenetic marks that confer a selective growth advantage or through a specific pathway initiated by one or more oncogenes (Jones, P. A. Cancer Res. 56, 2463-2467 (1996); Baylin, S. & Bestor, T. H. Cancer Cell 1, 299-305 (2002); Keshet, I. et al. Nat. Genet. 38, 149-153 (2006)). A better understanding of the mechanisms by which epigenetic gene silencing arises in cancer and identification of specific genes whose products affect this process would enable more efficacious therapeutics that selectively inhibit the epigenetic silencing pathways initiated by oncogene.

### SUMMARY OF INVENTION

Cancer development, or oncogenesis, is associated with the activation of oncogenes and inactivation of tumour suppressor and pro-apoptotic genes. These oncogenic changes often result from epigenetic gene silencing through hypermethylation of CpG-rich promoter regions. Identifying the key factors involved in this process of epigenetic gene silencing and understanding their roles oncogenesis would promote the discovery of cancer therapies that selectively inhibit epigenetic silencing pathways. We performed a genome-wide RNA interference (RNAi) screen to identify genes required for Ras-mediated epigenetic silencing of the pro-apoptotic Fas gene. Using K-ras transformed NIH 3T3 cells, we identified 28 genes required for Ras-mediated silencing of Fas that encode cell signalling molecules, chromatin modifiers, transcription factors, components of transcriptional repression complexes, and the DNA methyltransferase DNMT1. At least nine of these Ras epigenetic silencing effectors (RESEs), including DNMT1, are directly associated with specific regions of the Fas promoter in K-ras transformed NIH 3T3 cells but not in untransformed NIH 3T3 cells. RNAi-mediated knockdown of any of the 28 RESEs results in failure to recruit DNMT1 to the Fas promoter, loss of Fas promoter hypermethylation and de-repression of Fas expression. Analysis of five other epigenetically repressed genes indicates that Ras directs silencing of multiple, unrelated genes through a largely common pathway. Finally, we identify nine RESEs that are involved anchorage-independent growth and tumorigenicity of K-ras transformed NIH 3T3 cells; these nine genes have not been previously implicated in transformation by Ras. Our results demonstrate that Ras-mediated epigenetic silencing occurs through a specific unexpectedly complex pathway involving components that are required for maintenance of a fully transformed phenotype.

According to one aspect of the invention methods for inhibiting gene silencing in a cell are provided. The methods comprise reducing expression of one or more Ras epigenetic silencing effectors (RESEs) in the cell. In some embodiments the one or more RESEs are encoded by one or more genes of: KALRN, MAPK1, MAP3K9, PDPKI, PTK2B, S100Z, E1D1, CTCF, E2F1, RCOR2, SOX14, TRIM66, ZFP354B, BMI1, DNMT1, DOT1L, EED, EZH2, HDAC9, MRGBP, SMYD1, ASF1A, BAZ2A, NPM2, SIRT6, SIPA1L2, TRIM37, and ZCCHC4. In some embodiments the one or more RESEs are encoded by one or more genes of: NPM2, TRIM66, ZFP354B, BMI1, DNMT1, SIRT6, TRIM37, EZH2, and CTCF. In some embodiments the one or more RESEs are encoded by one or more genes of: BAZ2A, SMYD1, KALRN, S100Z, EID1, TRIM66, MRGBP, TRIM37, and ZCCHC4. In some embodiments the one or more RESEs are encoded by one or more genes of: KALRN, S100Z, EID1, TRIM66, MRGBP, TRIM37, and ZCCHC4. In some embodiments the methods provided are for inhibiting gene silencing, wherein the one or more the genes are one or more of: FAS, PAR4/MET, LOX, H2-K1, PLAGL1, and SFRP1. In some embodiments the methods provided are for inhibiting FAS gene silencing. In some embodiments methods are provided for inhibiting RAS dependent gene silencing. In some embodiments the inhibition of gene silencing comprises decreased DNA methylation. In certain embodiments the DNA methylation is mediated by DNMT1. In some embodiments the methods comprise reducing expression of one or more Ras epigenetic silencing effectors (RESEs), wherein the expression of RESEs is reduced by RNAi against the one or more mRNAs encoding the one or more RESEs. In certain embodiments the RNAi comprises contacting a cell with a composition comprising a siRNA molecule, shRNA molecule, shRNA-mir molecule, miRNA molecule, or dsRNA molecule. In certain other embodiments the RNAi comprises contacting a cell with a composition comprising a vector encoding a shRNA or shRNA-mir molecule.

According to one aspect of the invention methods for inhibiting silencing of a gene in a cell are provided, wherein the methods comprise reducing the interaction of one or more Ras epigenetic silencing effectors (RESEs) with a regulatory DNA sequence of the gene. In some embodiments the one or more RESEs are encoded by one or more genes of: NPM2, TRIM66, ZFP354B, BMI1, DNMT1, SIRT6, TRIM37, EZH2, and CTCF. In some embodiments the methods provided are for inhibiting gene silencing, wherein the one or more the genes are one or more of: FAS, PAR4/MET, LOX, H2-K1, PLAGL1, and SFRP1. In some embodiments the methods provided are for inhibiting FAS gene silencing. In some embodiments the interaction is reduced by RNAi against the one or more mRNAs encoding the one or more RESEs. In certain embodiments the RNAi comprises contacting the cell with a composition comprising a siRNA molecule, shRNA molecule, shRNA-mir molecule, miRNA molecule, or dsRNA molecule. In certain other embodiments the RNAi comprises contacting the cell with a composition comprising a vector encoding a shRNA or shRNA-mir molecule. In some embodiments the regulatory DNA sequence is located about at the transcriptional start site of the gene. In some embodiments the regulatory DNA sequence is within about 1 kb upstream of the transcriptional start site of the gene. In some embodiments the regulatory DNA sequence is within about 2 kb upstream of the transcriptional start site of the gene.

According to one aspect of the invention methods for inhibiting proliferation of a cell are provided. The methods comprise reducing expression of one or more Ras epigenetic silencing effectors (RESEs) in the cell. In some embodiments the one or more RESEs are encoded by one or more genes of: BAZ2A, SMYD1, KALRN, S100Z, EID1, TRIM66, MRGBP, TRIM37, and ZCCHC4. In some embodiments the one or more RESEs are encoded by one or more genes of: KALRN, S100Z, EID1, TRIM66, MRGBP, TRIM37, and ZCCHC4. In some embodiments the proliferation of the cell is RAS dependent. In some embodiments the proliferation of the cell is anchorage independent. In some embodiments the reducing expression comprises RNAi. In certain embodiments the RNAi comprises contacting a cell with a composition comprising a siRNA molecule, shRNA molecule, shRNA-mir molecule, miRNA molecule, or dsRNA molecule. In certain other embodiments the RNAi comprises contacting a cell with a composition comprising a vector encoding a shRNA or shRNA-mir molecule. In some embodiments the cell is in vitro. In some embodiments the cell is in vivo. In certain embodiments the cell forms a benign tumor. In certain other embodiments the cell forms a malignant tumor.

According to one aspect of the invention methods for inhibiting RAS-mediated growth of a tumor are provided. The methods comprise reducing expression of one or more Ras epigenetic silencing effectors (RESEs) in the tumor. In some embodiments the one or more RESEs are encoded by one or more genes of: BAZ2A, SMYD1, KALRN, S100Z, EID1, TRIM66, MRGBP, TRIM37, and ZCCHC4. In some embodiments the one or more RESEs are encoded by one or more genes of: KALRN, S100Z EID1, TRIM66, MRGBP, TRIM37, and ZCCHC4. In some embodiments the tumor is benign. In some embodiments the tumor is malignant. In certain embodiments the tumor is in a subject in need of a treatment that reduces the expression of the one or more RESEs in the cells comprised by the tumor. In some embodiments the reducing expression comprises RNAi. In certain embodiments the RNAi comprises contacting a cell with a composition comprising a siRNA molecule, shRNA molecule, shRNA-mir molecule, miRNA molecule, or dsRNA molecule. In certain other embodiments the RNAi comprises contacting a cell with a composition comprising a vector encoding a shRNA or shRNA-mir molecule. In certain embodiments the composition is a pharmaceutical composition.

According to one aspect of the invention methods for screening for regulators of FAS expression are provided. The methods comprise transducing eukaryotic cells with pools of a plurality of retroviruses, wherein individual retroviruses in the plurality comprises a nucleic acid encoding a product that modulates expression of at least one gene encoded in the genome of the transduced cells; isolating FAS positive transduced cells; and identifying the transduced nucleic acid. In some embodiments the isolating comprises selecting transduced cells containing a genomically integrated portion of the retroviral genome comprising the to nucleic acid. In certain embodiments the genomically integrated portion of the retroviral genome further comprises a sequence encoding a product that confers resistance to a compound. In certain embodiments the product that confers resistance to a compound is N-puromycin acetyltransferase. In certain embodiments the selecting comprises contacting the transduced cells with a compound that is inactivated by the product that confers resistance. In certain embodiments the compound is Puromycin. In some embodiments the isolating comprises immunoaffinity purification. In certain embodiments the immunoaffinity purification comprises contacting the transduced cells with an antibody or antigen binding fragment thereof that binds to FAS. In certain embodiments the identifying comprises isolating the genomically integrated portion of the retroviral genome comprising the nucleic acid. In certain embodiments the isolated nucleic acid is sequenced. In certain embodiments the product capable of affecting expression is an shRNA or shRNA-mir. In certain embodiments the shRNA or shRNA-mir is directed against the at least one gene encoded in the genome of the transduced cells. In certain embodiments the plurality of retroviruses comprise sequence complementary to a portion of the mRNA sequence of each of substantially all known protein coding genes of the transduced cell's genome.

According to one aspect of the invention methods for identifying compounds or compositions that inhibit RAS-mediated tumor growth are provided. The methods comprise contacting a cell with a compound or composition and assaying for decreased expression of one or more RESEs in the cell. In some embodiments the one or more RESEs are encoded by one or more genes of: KALRN, MAPK1, MAP3K9, PDPK1, PTK2B, S100Z, EID1, CTCF, E2F1, RCOR2, SOX14, TRIM66, ZFP354B, BMI1, DNMT1, DOT1L, EED, EZH2, HDAC9, MRGBP, SMYD1, ASF1A, BAZ2A, NPM2, SIRT6, SIPA1L2, TRIM37, and ZCCHC4. In some embodiments the methods further comprise assaying for altered expression of one or more of: FAS, PAR4/MET, LOX, H2-K1, PLAGL1, and SFRP1 in the cell. In some embodiments the methods further comprise assaying for altered DNA methylation at regulatory DNA sequences of one or more of: FAS, PAR4/MET, LOX, H2-K1, PLAGL1, and SFRP1 in the cell. In some embodiments the methods further comprise assaying for altered interaction of DNMT1 with regulatory DNA sequences of one or more of: FAS, PAR4/MET, LOX, H2-K1, PLAGL1, and SFRP1 in the cell.

These and other aspects of the invention, as well as various advantages and utilities, will be more apparent with reference to the detailed description of the invention. Each aspect of the invention can encompass various embodiments as will be understood by the following description.

### BRIEF DESCRIPTION OF DRAWINGS

FIG. 1 depicts the analysis of Fas gene expression in human cervical cancer HEC1A cells. A, Immunoblot analysis. HEC1A cells contain one normal and one activated Ras allele RasG12D). In HEC1AΔRasG12D cells, the activated Ras allele has been deleted (Kim, J. S., Lee, C., Foxworth, A. & Waldman, T. Cancer Res. 64, 1932-1937 (2004)). Fas expression was monitored in HEC1A cells, in HEC1AΔRasG12D cells and in HEC1A cells treated with 5-aza. Actin was monitored as a loading control. B, Quantitative real-time RT-PCR (qRT-PCR) analysis monitoring Fas expression. Error bars indicate standard error.

FIG. 2 depicts a genome-wide shRNA screen that identifies factors required for Ras-mediated epigenetic silencing of Fas. A, Depicts a schematic summary of the genome-wide shRNA screen for Ras-mediated epigenetic silencing of Fas. B, Depicts immunoblot analysis monitoring Fas expression in the 28 K-Ras NIH 3T3 knockdown (KD) cell lines. Expression of Fas in K-Ras NIH 3T3 cells in the presence and absence of 5-aza-2′-deoxycytidine (5-aza) is also shown. K-Ras expression is shown as a loading control.

FIG. 3 depicts an analysis of target gene expression in the K-Ras NIH 3T3 KD cell lines. Quantitative real-time RT-PCR (qRT-PCR) was used to analyze target gene expression in each of the 28 K-Ras NIH 3T3 KD cell lines. Error bars indicate standard error.

FIG. 4 depicts confirmation of all 28 RESEs using a second, unrelated shRNA directed against the target gene. qRT-PCR analysis shows that a second, unrelated shRNA directed against the target gene also resulted in Fas re-expression (top) and decreased expression of the target gene (bottom). NS, nonsilencing shRNA. Error bars indicate standard error.

FIG. 5 illustrates the knockdown of the 28 RESEs in a second, unrelated cell line, H-Ras transformed murine C3H10T1/2 fibroblasts, results in Fas re-expression. A, qRT-PCR analysis reveals that knockdown of each of the 28 RESEs resulted in Fas re-expression (top) and decreased expression of the target gene (bottom) in C3H10T1/2 cells. NS, nonsilencing shRNA. Error bars indicate standard error. B, Bisulphite sequencing analysis of Fas. Each circle represents a CpG dinucleotide. Open (white) circles denote unmethylated CpG sites; filled (black) circles indicate methylated CpG sites. Each row represents a single clone; for each cell line, six clones were sequenced. The regions of the promoter analyzed are shown. The position of the transcription start-site is indicated by the arrow, and positions of the CpG dinucleotides are shown to scale by vertical lines.

FIG. 6 shows that several RESEs are upregulated at the transcriptional level in K-Ras NIH 3T3 cells. Quantitative real-time RT-PCR (qRT-PCR) was used to analyze RESE gene expression in K-Ras NIH 3T3 cells compared to NIH 3T3 cells. Values are expressed as fold upregulation in K-Ras NIH 3T3 cells relative to expression in NIH 3T3 cells. Error bars indicate standard error.

FIG. 7 demonstrates that ZFP354B is upregulated at the post-transcriptional level by K-Ras. A, Immunoblot analysis showing up-regulation of ZFP354B protein expression in K-Ras NIH 3T3 cells compared to NIH 3T3 cells. Addition of the phosphoinositide-3 kinase (PI3K) inhibitor LY294002 prevented upregulation of ZFP354B; PI3K is a downstream effector of Ras. ZFP354B upregulation was also abrogated upon treatment with an shRNA directed against the kinase PDPK1, a RESE (see Tables 1 and 2) and known downstream effector of Ras, or ZFP354B itself, but not a nonsilencing (NS) control shRNA. Endogenous ZFP354B was monitored using an antiZFP354B antibody, and tubulin was monitored as a loading control using an anti-tubulin antibody. B, Quantitative real-time RT-PCR (qRT-PCR) was used to analyze Zfp354b gene expression in K-Ras NIH 3T3 cells compared to NIH 3T3 cells. The results reveal that Zfp354b is not transcriptionally upregulated in K-Ras NIH 3T3 cells compared to NIH 3T3 cells. Error bars indicate standard error. C, Immunoblot analysis. Plasmids expressing activated K-Ras and/or C-terminal V5tagged ZFP354B or a mutant derivative lacking the N-terminal PEST sequence [ZFP354B (ΔPEST)] were cotransfected into COS cells and 36 hours laters cells were harvested for immunoblot analysis. ZFP354B was monitored using an anti-V5 antibody, and tubulin was monitored as a loading control using an anti-tubulin antibody. The results show that ZFP354B protein levels increased in the presence of Ras, and that this increase depended on the presence of the PEST sequence, an element known to be involved in regulated protein stability.

FIG. 8 illustrates a ChIP analysis and methylation status of the Fas promoter. A, Summary of bisulphite sequencing analysis of the Fas promoter in NIH 3T3 and K-ras NIH 3T3 cells, and in K-ras NIH 3T3 cells in which DNMT1 is knocked down by shRNA treatment. Each circle represents a CpG dinucleotide: open (white) circles denote unmethylated CpG sites and filled (black) circles indicate methylated CpG sites. Each row represents a single clone; for each cell line six clones were sequenced. Positions of the CpG dinucleotides are shown to scale by vertical lines. The position of the first exon and intron are shown in grey. B, Methylated DNA immunoprecipitation (MeDIP) assay of the Fas promoter, using primer-pairs corresponding to the TSS/DS region as shown in the schematic. C, MeDIP analysis of the Fas hypermethylated regions following knockdown of each of the 28 RESEs. NS, nonsilencing shRNA. Values are expressed as the fold-difference relative to input, and have been corrected for background. D, Chromatin immunoprecipitation (ChIP) assay monitoring Fas promoter occupancy of a subset of the 28 Ras epigenetic silencing effectors (RESEs). Primer-pairs located at the core promoter/TSS(CP/TSS), ˜1 kb upstream of the TSS (˜1 kb) or ˜2 kb upstream of the TSS (˜2 kb) were used for PCR analysis of the input and immunoprecipitated DNA samples. E, Summary of the ChIP results on the Fas promoter in NIH 3T3 and K-ras NIH 3T3 cells. F, ChIP analysis monitoring occupancy of DNMT1 on the Fas promoter following knockdown of each of the 28 RESEs. Values are expressed as the fold-difference relative to input, and have been corrected for background.

FIG. 9 illustrates that DNA methyltransferases DNMT3A and DNMT3B do not detectably associate with the Fas promoter. Chromatin immunoprecipitation (ChIP) monitoring Fas promoter occupancy of DNMT3A and DNMT3B at the CP/TSS, ˜1 kb upstream of the TSS, ˜2 kb upstream of the TSS. As a control, binding of DNMT3A and DNMT3B was also monitored at the gamma satellite region, a known target of DNMT3A and DNMT3B3. Values are expressed as the fold-enrichment relative to input, and have been corrected for background. Error bars indicate standard error.

FIG. 10 depicts that Ras directs epigenetic silencing of multiple, unrelated genes through a largely common pathway. A, Quantitative RT-PCR (qRT-PCR) monitoring expression of Fas, Sfrp1, Par4, Plagl1, H2-K1 and Lox in NIH 3T3 cells, and in K-ras NIH 3T3 cells in the presence and absence of 5-aza. Values are expressed as fold re-expression relative to expression of the gene in K-ras NTH 3T3 cells, which is arbitrarily set to 1. B, Bisulphite sequencing analysis of the Sfrp1 promoter. C, Summary of qRT-PCR analysis monitoring re-expression of Fas, Sfrp1, Par4, Plagl1, H2-K1 and Lox following knockdown of each of the 28 RESEs. D, MeDIP analysis of the Sfrp1 hypermethylated region following knockdown of each of the 28 RESEs.

FIG. 11 depicts hypermethylation of Par4, Plagl1, H2-K1, and Lox in K-ras NIH 3T3 cells using bisulphite sequencing analysis. Each circle represents a CpG dinucleotide. Open (white) circles denote unmethylated CpG sites; filled (black) circles indicate methylated CpG sites. Each row represents a single clone; for each cell line, six clones were sequenced. The region(s) of the promoters analyzed is shown. The position of the transcription start-site is indicated by the arrow, and positions of the CpG dinucleotides are shown to scale by vertical lines. Exons and introns are indicated by gray thick and thin lines, respectively.

FIG. 12 illustrates that Ras directs epigenetic silencing of multiple, unrelated genes through a largely common pathway. Quantitative real-time RT-PCR (qRT-PCR) analysis monitoring re-expression of Fas, Par4, Lox, H2-K1, Plagl1 and Sfrp1 following knockdown of each of the 28 RESEs. NS, nonsilencing shRNA. Values are expressed as fold re-expression relative to expression of the gene in K-Ras NIH 3T3 cells. The red line indicates 2-fold re-expression. Error bars indicate standard error.

FIG. 13 illustrates the requirement of factors involved in Ras-mediated epigenetic silencing for a fully transformed phenotype. A, Soft agar growth assay. The 28 K-Ras NIH 3T3 KD cell lines were tested for their ability to grow in soft agar. NS, nonsilencing shRNA. Values are expressed as percentage growth relative to parental K-Ras NIH 3T3 cells. B, Tumour formation assay. Each of the indicated K-Ras NIH 3T3 KD cell lines was subcutaneously injected into the flanks of nude mice, and tumour volume was measured every 3 days for 15 days (n=3 mice per time point). Error bars indicate standard error.

FIG. 14 depicts MeDIP analysis of the Par4, Plagl1, H2-K1, and Lox hypermethylated regions following knockdown of each of the 28 RESEs. MeDIP analysis following knockdown of each of the 28 RESEs. NS, nonsilencing shRNA. Values are expressed as the fold-difference relative to input, and have been corrected for background.

### DETAILED DESCRIPTION

The conversion of a normal cell to a cancer cell is a stepwise process that typically involves the activation of oncogenes and inactivation of tumor suppressor and pro-apoptotic genes. In many instances, inactivation of genes critical for cancer development occurs by epigenetic silencing that often involves hypermethylation of CpG-rich promoter regions. Members of the Ras oncogene family transform most immortalized cell lines in vitro, and mutations of Ras genes occur in ˜30% of cancer-related human tumors (Giehl, K. Oncogenic Ras in tumour progression and metastasis. Biol. Chem. 386, 193-205 (2005)). In addition, activation of the Ras pathway is frequent in human tumors even in the absence of Ras mutations (Ehmann, F. et al. Leuk. Lymphoma 47, 1387-1391 (2006)). Previous studies have shown that in mouse NIH 3T3 cells activated Ras epigenetically silences Fas expression thereby preventing Fas-ligand induced apoptosis (Fenton, R. G., Hixon, J. A., Wright, P. W., Brooks, A. D. & Sayers, T. J. Cancer Res. 58, 3391-3400 (1998); Peli, J. et al. EMBO J. 18, 1824-1831 (1999)). In addition, epigenetic silencing of Fas occurs in some transformed cells, human tumors, and mouse models of cancer, and this silencing is relevant to tumor progression (see, for example, Hopkins-Donaldson, S. et al. Cell Death Differ. 10, 356-364 (2003)).

A new strategy for the systematic identification of genes required for Ras-mediated silencing of Fas is disclosed herein. In one aspect, a genome-wide small hairpin RNA (shRNA) screen is used to identify genes involved in Ras-mediated epigenetic silencing of the pro-apoptotic Fas gene. Using K-ras transformed NIH 3T3 cells, a plurality of genes are identified that are involved in Ras-mediated silencing of Fas and that encode cell signalling molecules, chromatin modifiers, transcription factors, components of transcriptional repression complexes, and the DNA methyltransferase DNMT1. At least nine of these Ras epigenetic silencing effectors (RESEs), including DNMT1, are directly associated with specific regions of the Fas promoter in K-ras transformed NIH 3T3 cells but not in untransformed NIH 3T3 cells. RNAi-mediated knockdown of any of the plurality of RESEs results in failure to recruit DNMT1 to the Fas promoter, loss of Fas promoter hypermethylation and de-repression of Fas expression. Analysis of five other epigenetically repressed genes indicates that Ras directs silencing of multiple, unrelated genes through a largely common pathway. In one aspect, nine RESEs are discovered to be involved in anchorage-independent growth and tumorigenicity of K-ras transformed NIH 3T3 cells; these nine genes have not been previously implicated in transformation by Ras. Certain aspects demonstrate that Ras-mediated epigenetic silencing occurs by a specific, unexpectedly complex pathway involving components that are involved in the maintenance of a fully transformed phenotype.

As used herein, “suppress”, “inhibit”, or “reduce” may, or may not, be complete. For example, cell proliferation may, or may not, be decreased to a state of complete arrest for an effect to be considered one of suppression or inhibition. Similarly, gene expression may, or may not, be decreased to a state of complete cessation for an effect to be considered one of suppression or reduction. Moreover, “suppress”, “inhibit”, or “reduce” may comprise the maintenance of an existing state and the process of affecting a state change. For example, inhibition of cell proliferation may refer to the prevention of proliferation of a non-proliferating cell (maintenance of a non-proliferating state) and the process of inhibiting the proliferation of a proliferating cell (process of affecting a proliferation state change). Similarly, inhibition of gene silencing may refer to the prevention of silencing of a non-silenced (e.g., expressed) gene (maintenance of an expressed state) and the process of ceasing the silencing (e.g., activating) of a silenced gene (process of affecting a gene expression state change).

In one embodiment, a cell culture system is used to screen for RAS-mediated epigenetic gene silencing effector genes (See Examples). The system provides an assay for cell surface expression or re-expression of Fas. In one embodiment Fas-positive cells are selected on immunomagnetic beads using an anti-Fas antibody and expanded in culture. The model system provides test cells and control cells. As described herein, test or control cells can be primary cells, non-immortalized cell lines, immortalized cell lines, transformed immortalized cell lines, benign tumor derived cell lines, malignant tumor derived cell lines, or transgenic cell lines. More than one set of control cells may be provided, such as non-Ras transformed and Ras transformed cell lines. Cells in this system may be subjected to one or more genetic or chemical perturbations and then incubated for a predetermined time. The predetermined time is a time sufficient to produce a desired effect (e.g., Fas re-expression) in a control cell.

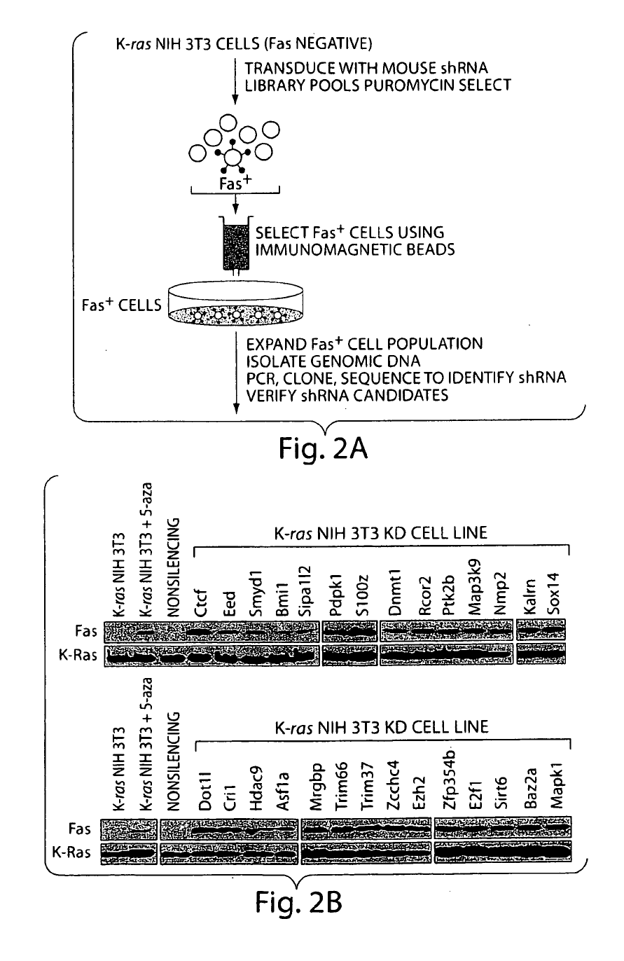

In one embodiment, the cell culture system disclosed herein is used to screen for RAS-mediated epigenetic gene silencing effector genes (i.e., effectors) in systematic and efficient manner. In one embodiment, the screen combines RNAi mediated gene suppression with an assay for Ras mediated epigenetic gene silencing of Fas. This embodiment involves a genome-wide RNAi based genetic screen using, as a selection strategy, re-expression of Fas protein on the cell surface (FIG. 2a). The methods of this screen are applicable to the use of libraries comprising RNAi based modalities consisting of from a single gene to all, or substantially all, known genes in an organism under investigation. In one embodiment, a mouse shRNA-mir library comprising about 62,400 shRNA-mirs directed against about 28,000 genes was divided into 10 pools, which were packaged into retrovirus particles and used to stably transduce Fas-negative, K-Ras NIH 3T3 cells (See Examples). Methods for viral packaging and transduction of cells, including those described herein, are well known to one of ordinary skill in the art.

In a preferred embodiment, the library utilizes a mir-30-based shRNA (shRNAmir) expression vector in which shRNA sequence is flanked by approximately 125 bases 5′ and 3′ of the pre-miR-30 sequence (Chang K, Elledge S J, Hannon G J. Nat. Methods. 2006 Sep.; 3(9):707-14.). Expression vectors can employ either polymerase II or polymerase III promoters to drive expression of these shRNAs and result in functional siRNAs in cells. The former polymerase permits the use of classic protein expression strategies, including inducible and tissue-specific expression systems. Other library compilations, such Lentiviral-based systems and libraries directed against human sequences, are readily available and well known to one of ordinary skill in the art.

An expression vector is one into which a desired sequence may be inserted, e.g., by restriction and ligation, such that it is operably joined to regulatory sequences and may be expressed as an RNA transcript. An expression vector typically contains an insert that is a coding sequence for a protein or for a functional RNA such as an shRNA, a miRNA, or an shRNA-mir. Vectors may further contain one or more marker sequences suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays known in the art (e.g., (β-galactosidase or alkaline phosphatase), and genes that visibly affect the phenotype of transformed or transfected cells, hosts, colonies or plaques (e.g., green fluorescent protein).

As used herein, a coding sequence (e.g., protein coding sequence, miRNA sequence, shRNA sequence) and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide. It will be appreciated that a coding sequence need not encode a protein but may instead, for example, encode a functional RNA such as an miRNA, shRNA or shRNA-mir.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Such 5′ non-transcribed regulatory sequences will include a promoter region that includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art. Exemplary regulatory sequences for expression of interfering RNA (e.g., shRNA, miRNA) are disclosed herein. One of skill in the art will be aware of these and other appropriate regulatory sequences for expression of interfering RNA, e.g., shRNA, miRNA, etc.

In some embodiments, a virus vector for delivering a nucleic acid molecule is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), lentiviral vectors (Naldini L, et al., Proc Natl Acad Sci USA. 1996 Oct. 15; 93(21):11382-8) and Ty virus-like particle (Allsopp et al., Eur. J. Immunol 26:1951-1959, 1996).

Another virus useful for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other useful viral vectors are based on non-cytopathic eukaryotic viruses in which non-essential genes have been replaced with the gene of interest. Non-cytopathic viruses include certain retroviruses, the life cycle of which involves reverse transcription of genomic viral RNA into DNA with subsequent proviral integration into host cellular DNA. In general, the retroviruses are replication-deficient (i.e., capable of directing synthesis of the desired transcripts, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Clifton, N.J. (1991).

Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like. Other examples include: N-TER™ Nanoparticle Transfection System by Sigma-Aldrich, FectoFly™ transfection reagents for insect cells by Polyplus Transfection, Polyethylenimine “Max” by Polysciences, Inc., Unique, Non-Viral Transfection Tool by Cosmo Bio Co., Ltd., Lipofectamine™ LTX Transfection Reagent by Invitrogen, SatisFection™ Transfection Reagent by Stratagene, Lipofectamine™ Transfection Reagent by Invitrogen, FuGENE® HID Transfection Reagent by Roche Applied Science, GMP compliant in vivo-jetPEI™ transfection reagent by Polyplus Transfection, and Insect GeneJuice® Transfection Reagent by Novagen.

In one embodiment the cell culture system disclosed herein is used to screen for RAS-mediated epigenetic gene silencing effector genes (i.e., effectors), wherein the screen combines cDNA-based exogenous gene expression with an assay for Ras mediated epigenetic gene silencing of Fas. This embodiment involves a genome-wide cDNA based genetic screen using, as a selection strategy, re-expression of Fas protein on the cell surface. The methods of this screen are applicable to the use of libraries comprising cDNA based modalities consisting of from a single gene to all, or substantially all, known genes in an organism under investigation.

In one embodiment, experimental systems are contemplated in which a large set of samples, such as the genome-wide shRNA-mir library disclosed herein, is screened without pooling. Such systems make use of high-throughput biological techniques and equipment, such as laboratory automation and sample tracking processes well known to one of ordinary skill in the art. In such systems, other non-vector based libraries (e.g., siRNA libraries) could be screened. Thus, the assay methods of the invention are amenable to high-throughput screening (HTS) implementations. In some embodiments, the screening assays of the invention are high throughput or ultra high throughput (e.g., Fernandes, P. B., Curr Opin Chem. Biol. 1998 2:597; Sundberg, S A, Curr Opin Biotechnol. 2000, 11:47). HTS refers to testing of up to, and including, 100,000 compounds or compositions per day, whereas ultra high throughput (uHTS) refers to screening in excess of 100,000 compounds or compositions per day. The screening assays of the invention may be carried out in a multi-well format, for example, a 6-well, 12-well, 24-well, 96-well, 384-well format, or 1,536-well format, and are suitable for automation. In the high throughput assays of the invention, it is possible to screen several thousand different compounds or compositions in a single day. In particular, each well of a microtiter plate can be used to run a separate assay against a selected test compound or composition, or, if concentration or incubation time effects are to be observed, a plurality of wells can contain test samples of a single compound or composition. It is possible to assay many plates per day; assay screens for up to about 6,000, 20,000, 50,000, or more than 100,000 different compounds are possible using the assays of the invention. Typically, HTS implementations of the assays disclosed herein involve the use of automation. In some embodiments, an integrated robot system consisting of one or more robots transports assay microplates between multiple assay stations for compound, cell and/or reagent addition, mixing, incubation, and finally readout or detection. In some aspects, an HTS system of the invention may prepare, incubate, and analyze many plates simultaneously, further speeding the data-collection process. High throughput screening implementations are well known in the art. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jörg Hüser, the contents of which are both incorporated herein by reference in their entirety.

The methods described herein have broad application to disorders, such as cancer, that are associated with alteration of Ras-mediated epigenetic silencing effectors. Cancer is disease characterized by uncontrolled cell proliferation and other malignant cellular properties. As used herein, the term cancer includes, but is not limited to, the following types of cancer: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will be known to one of ordinary skill in the art.

Cell transformation can arise from a number of genetic and epigenetic perturbations that cause defects in mechanisms controlling cell migration, proliferation, differentiation, and growth. As used herein, transformation describes the conversion of a cell from a non-tumorigenic to a tumorigenic state and resulting tumors can be either benign or malignant. Whereas benign tumors remain localized in a primary tumor that remains localized at the site of origin and that is often self limiting in terms of tumor growth, malignant tumors have a tendency for sustained growth and an ability to spread or metastasize to distant locations. Malignant tumors develop through a series of stepwise, progressive changes that lead to uncontrolled cell proliferation and an ability to invade surrounding tissues and metastasize to different organ sites.

As disclosed herein, one aspect of the treatment methods of the invention contemplates treatment of a subject having or at risk of having a Ras-dependent tumor. As used herein, a subject is a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), zoo animals (e.g., lions, giraffes, etc.), but are not so limited. Preferred subjects are human subjects. The human subject may be a pediatric, adult or a geriatric subject.

In some embodiments, the methods involve treating a subject in need thereof by administering a compound or composition (e.g., an RNAi molecule) that inhibits Ras dependent tumor formation and/or growth. In some embodiments, the compound or composition reduces the expression of one or more Ras epigenetic silencing effectors (RESEs) in cells of the tumor and inhibits growth of the tumor. In some embodiments, the compound or composition reduces the expression of one or more of KALRN, MAPK1, MAP3K9, PDPK1, PTK2B, S100Z, EID1, CTCF, E2F1, RCOR2, SOX14, TRIM66, ZFP354B, BMI1, DNMT1, DOT1L, EED, EZH2, HDAC9, MRGBP, SMYD1, ASF1A, BAZ2A, NPM2, SIRT6, SIPA1L2, TRIM37, and ZCCHC4.

As used herein treatment or treating includes amelioration, cure or maintenance (i.e., the prevention of relapse) of a disorder (e.g, a Ras-dependent tumor). Treatment after a disorder has started aims to reduce, ameliorate or altogether eliminate the disorder, and/or its associated symptoms, to prevent it from becoming worse, or to prevent the disorder from re-occurring once it has been initially eliminated (i.e., to prevent a relapse).

As used herein, a therapeutically effective amount is an amount of a compound or composition (e.g., an RNAi molecule) that inhibits Ras dependent tumor formation and/or growth and/or that reduces expression of one or more Ras epigenetic silencing effectors to produce a therapeutically beneficial result. A therapeutically effective amount can refer to any compounds or compositions described herein, or discovered using the methods described herein, that have Ras-dependent tumor inhibitory properties (e.g, inhibit the growth of Ras-transformed cells). The therapeutically effective amount of the active agent to be included in pharmaceutical compositions depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg. One of ordinary skill in the art would be able to determine empirically an appropriate therapeutically effective amount. Methods for establishing a therapeutically effective amount for any compounds or compositions described herein will be known to one of ordinary skill in the art. As used herein, pharmacological compositions comprise compounds or compositions that have therapeutic utility, and a pharmaceutically acceptable carrier, i.e., that facilitate delivery of compounds or compositions, in a therapeutically effective amount.

The disclosure in other embodiments provides a pharmaceutical pack or kit comprising one or more containers filled with one or more of the ingredients of the pharmaceutical compositions of the invention. Associated with such container(s) can be various written materials (written information) such as instructions (indicia) for use, or a notice in the form prescribed by a governmental agency regulating the manufacture, use or sale of pharmaceuticals or biological products, which notice reflects approval by the agency of manufacture, use or sale for human administration.

As used herein, the term “pharmaceutically acceptable carrier” includes any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the compositions of this invention, its use in the therapeutic formulation is contemplated. Supplementary active ingredients can also be incorporated into the pharmaceutical formulations. A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. See, for example, REMINGTON'S PHARMACEUTICAL SCIENCES, 18th Ed. (1990).

It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

The pharmaceutical compositions of the present invention preferably contain a pharmaceutically acceptable carrier or excipient suitable for rendering the compound or mixture administrable orally as a tablet, capsule or pill, or parenterally, intravenously, intradermally, intramuscularly or subcutaneously, or transdermally. The active ingredients may be admixed or compounded with any conventional, pharmaceutically acceptable carrier or excipient.

The pharmaceutical compositions disclosed herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or as an aerosol. Depending upon the type of condition (e.g., cancer) to be treated, compounds of the invention may, for example, be inhaled, ingested or administered by systemic routes. Thus, a variety of administration modes, or routes, are available. The particular mode selected will depend, of course, upon the particular compound or composition selected, the particular condition being treated and the dosage required for therapeutic efficacy. The methods, generally speaking, may be practiced using any mode of administration that is medically acceptable, meaning any mode that produces acceptable levels of efficacy without causing clinically unacceptable adverse effects. Preferred modes of administration are parenteral and oral routes. The term “parenteral” includes subcutaneous, intravenous, intramuscular, intraperitoneal, and intrasternal injection, or infusion techniques.

As used herein, gene therapy is a therapy focused on treating genetic diseases, such as cancer, by the delivery of one or more expression vectors encoding therapeutic gene products, including polypeptides or RNA molecules, to diseased cells. In one embodiment a composition capable of sufficiently and substantially inhibiting Ras dependent tumor formation and/or the growth of Ras-transformed cells is a gene therapy comprising an expression vector, wherein the expression vector preferable encodes one or more molecules (e.g., an shRNA) that specifically suppress the expression of one or more RESEs, preferably one or more of the RESEs in Tables 1 and 2. Methods for construction and delivery of expression vectors are disclosed herein and will be known to one of ordinary skill in the art.

In one embodiment, reduction of the interaction of a RESE with a regulatory DNA sequence of a Ras regulated gene in a cell provides a method for inhibiting silencing of the Ras regulated gene. In one embodiment, reduction of the interaction of a RESE with a regulatory DNA sequence of a Ras regulated gene in a cell provides a method for inhibiting proliferation of the cell. In one embodiment, reduction of the interaction of a RESE with a regulatory DNA sequence of a Ras regulated gene in a cell provides a method for inhibiting growth of a tumor comprising the cell.

Approaches known to one of ordinary skill in the art can be employed to reduce the binding of a RESE with a regulatory DNA sequence of a Ras regulated gene. For example, exogenous expression of a DNA-binding domain fragment of a DNA-binding RESE could competitively inhibit binding of the corresponding full-length RESE to a regulatory DNA sequence of a Ras regulated gene, thereby reducing the interaction of the RESE with the regulatory DNA sequence of the Ras regulated gene. In certain embodiments, inhibition of expression a RESE reduces the interaction of the RESE with a regulatory DNA sequence of a Ras regulated gene.

In one embodiment, inhibition of expression of a RESE gene in a cell provides a method for inhibiting silencing of a Ras regulated gene in the cell. In one embodiment, inhibition of expression of a RESE gene in a cell provides a method for inhibiting proliferation of the cell. In one embodiment, inhibition of expression of a RESE gene in a cell provides a method for inhibiting growth of a tumor comprising the cell. The expression of an RESE gene can be inhibited using various strategies for gene knockdown known in the art. For example gene knockdown strategies that make use of RNA interference (RNAi) and/or microRNA (miRNA) pathways including small interfering RNA (siRNA), short hairpin RNA (shRNA), or double-stranded dsRNA, miRNAs, or other nucleotide-based molecules can be used. In one embodiment, vector-based RNAi modalities (e.g., shRNA or shRNA-mir expression constructs) are used to reduce expression of an RESE in a cell.

TABLE 1
Ras epigenetic silencing effector (RESEs) and Reporter Gene Identifiers

| Official Gene | | | | |
|---|---|---|---|---|
| Symbol (Human) | Gene Aliases | Human GeneID | Mouse GeneID | Homologue ID |
| ASF1A | RP3-329L24.1, CGI-98, CIA, | 25842 | 66403 | 8528 |
| | DKFZP547E2110, HSPC146 | | | |
| BAZ2A | DKFZp781B109, FLJ13768, | 11176 | 116848 | 8393 |
| | FLJ13780, FLJ45876, | | | |
| | KIAA0314, TIP5, WALp3 | | | |
| BMI1 | RP11-573G6.1, MGC12685, | 164831 | 12151 | 3797 |
| | PCGF4, RNF51 | | | |
| CTCF | | 10664 | 13018 | 4786 |
| C20orf20 | Eaf7, FLJ10914, MRG15BP, | 55257 | 73247 | 10104 |
| | MRGBP | | (1600027N09Rik) | |
| DNMT1 | CXXC9, DNMT, FLJ16293, | 126375 | 13433 | 1055 |
| | MCMT, MGC104992 | | | |
| DOT1L | DOT1, KIAA1814 | 84444 | 208266 | 32779 |
| EED | HEED, WAIT1 | 8726 | 13626 | 2814 |
| EID1 | C15orf3, CRI1, EID-1, | 23741 | 58521 | 49376 |
| | IRO45620, MGC138883, | | | |
| | MGC138884, PNAS-22, | | | |
| | PTD014, RBP21 | | | |
| EZH2 | ENX-1, EZH1, MGC9169 | 2146 | 14506 | 37926 |
| E2F1 | E2F-1, RBBP3, RBP3 | 1869 | 13555 | 3828 |
| HDAC9 | DKFZp779K1053, HD7, HDAC, | 9734 | 79221 | 64351 |
| | HDAC7, HDAC7B, HDAC9B, | | | |
| | HDAC9FL, HDRP, KIAA0744, | | | |
| | MITR | | | |
| KALRN | DUET, FLJ16443, HAPIP, | 8997 | 545156 | 57160 |
| | TRAD, duo | | | |
| MAPK1 | ERK, ERK2, ERT1, MAPK2, | 5594 | 26413 | 37670 |
| | P42MAPK, PRKM1, PRKM2, | | | |
| | p38, p40, p41, p41mapk | | | |
| MAP3K9 | MLK1, PRKE1 | 4293 | 338372 | 76377 |
| NPM2 | MGC78655 | 10361 | 328440 | 15349 |
| PDPK1 | MGC20087, MGC35290, | 5170 | 18607 | 37643 |
| | PDK1, PRO0461, PkB-like, | | | |
| | PkB-like 1 | | | |
| PTK2B | CADTK, CAKB, FADK2, FAK2, | 2185 | 19229 | 23001 |
| | PKB, PTK, PYK2, RAFTK | | | |
| RCOR2 | | 283248 | 104383 | 14280 |
| SIPA1L2 | FLJ23126, FLJ23632, | 57568 | 244668 | 18956 |
| | KIAA1389, SPAL2 | | | |
| SIRT6 | SIR2L6 | 51548 | 50721 | 6924 |
| SMYD1 | BOP, ZMYND18, ZMYND22 | 150572 | 12180 | 7645 |
| SOX14 | MGC119898, MGC119899, | 8403 | 20669 | 31224 |
| | SOX28, SRY-box 14 | | | |
| S100Z | Gm625, S100-zeta | 170591 | 268686 | 15633 |
| TRIM37 | KIAA0898, MUL, POB1, TEF3 | 4591 | 68729 | 9084 |
| TRIM66 | TIF1D, TIF1DELTA | 9866 | 330627 | 28044 |
| ZCCHC4 | HSPC052, MGC21108 | 29063 | 78796 | 14632 |
| ZNF354B | FLJ25008, KID2, MGC138316 | 117608 | 27274 | 32187 |
| | | | (Zfp354b) | |
| FAS | ALPS1A, APO-1, APT1, CD95, | 355 | 14102 | 27 |
| | FAS1, FASTM, TNFRSF6 | | | |
| HLA-G | DAQB-346J13.1, MHC-G | 3135 | 14972 | 90872 |
| | | | (H2-K1) | |
| LOX | MGC105112 | 4015 | 16948 | 1741 |
| PAWR | PAR4, Par-4 | 5074 | 114774 | 1940 |
| PLAGL1 | DKFZp781P1017, LOT1, | 5325 | 22634 | 31401 |
| | MGC126275, MGC126276, | | | |
| | ZAC, ZAC1 | | | |
| SFRP1 | FRP, FRP-1, FRP1, FrzA, | 6422 | 20377 | 2266 |
| | SARP2 | | | |

TABLE 2
Ras-Mediated Epigenetic Silencing Effector Genes

| Biological process | Accession number | Gene symbol | Name |
|---|---|---|---|
| Signal transduction | XM_993034 | Kalrn | kalirin, RhoGEF kinase |
| | NM_011949 | Mapk1 | mitogen-activated protein kinase 1 |
| | NM_177395 | Map3k9 | mitogen-activated protein kinase kinase kinase 9 |
| | NM_011062 | Pdpk1 | 3-phosphoinositide dependent protein kinase 1 |
| | NM_172498 | Ptk2b | PTK2 protein tyrosine kinase 2 beta |
| | XM_193738 | S100z | S100 calcium binding protein, zeta |
| Transcription regulation | NM_025613 | Eid1 | EP300 interacting inhibitor of differentiation 1 |
| | NM_181322 | Ctcf | CCCTC-binding factor |
| | NM_007891 | E2f1 | E2F transcription factor 1 |
| | NM_054048 | Rcor2 | REST corepressor 2 |
| | XM_284529 | Sox14 | SRY -box containing gene 14 |
| | NM_181853 | Trim66 | tripartite motif-containing protein 66 |
| | NM_013744 | Zfp354b | zinc finger protein 354B |
| Chromatin modification | NM_007552 | Bmi1 | Bmi1 polycomb ring finger oncogene |
| | NM_010066 | Dnmt1 | DNA methyltransferase (cytosine-5) 1 |
| | NM_199322 | Dot1l | DOT1-like, histone H3 methyltransferase |
| | NM_021876 | Eed | embryonic ectoderm development |
| | NM_007971 | Ezh2 | enhancer of zeste homolog 2 |
| | NM_024124 | Hdac9 | histone deacetylase 9 |
| | NM_028479 | Mrgbp | MRG binding protein |
| | NM_009762 | Smyd1 | SET and MYND domain containing 1 |
| Chromatin remodeling | NM_025541 | Asf1a | ASF1 anti-silencing function 1 homolog A (S. |
| | | | cerevisiae) |
| | NM_054078 | Baz2a | bromodomain adjacent to zinc finger domain, 2A |
| | NM_181345 | Npm2 | nucleophosmin/nucleoplasmin, 2 |
| Genome stability/Aging | NM_181586 | Sirt6 | sirtuin 6 (silent mating type information regulation |
| | | | 2, homolog) 6 (S. cerevisiae) |
| Unknown | XM_146572 | Sipa1l2 | signal-induced proliferation-associated gene 1 like 2 |
| | NM_197987 | Trim37 | tripartite motif-containing protein 37 |
| | XM_132052 | Zcchc4 | zinc finger, CCHC domain containing 4 |

A broad range of other RNAi-based modalities could be also employed to reduce expression of an RESE in a cell (for example, to treat a subject having or at risk of having a Ras-dependent tumor), such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluoyl nucleotides maintain gene silencing activity (Xia et al., ASC Chem. Biol. 1(3):176-83, (2006)). siRNAs with amide-linked oligoribonucleosides have been generated that are more resistant to S1 nuclease degradation than unmodified siRNAs (Iwase R et al. 2006 Nucleic Acids Symp Ser 50: 175-176). In addition, modification of siRNAs at the 2′-sugar position and phosphodiester linkage confers improved serum stability without loss of efficacy (Choung et al., Biochem. Biophys. Res. Commun. 342(3):919-26, 2006). Moreover, 2′-deoxy-2′-fluoro-beta-D-arabinonucleic acid (FANA)-containing antisense oligonucleotides compared favourably to phosphorothioate oligonucleotides, 2′-O-methyl-RNA/DNA chimeric oligonucleotides and siRNAs in terms of suppression potency and resistance to degradation (Ferrari N et al. 2006 Ann N Y Acad Sci 1082: 91-102).

Other molecules that can be used include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat. Med. 4(8):967-71, 1998). For example, neoplastic reversion was obtained using a ribozyme targeted to an H-Ras mutation in bladder carcinoma cells (Feng et al., Cancer Res. 55(10):2024-8, 1995). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9,1996). Multitarget ribozymes (connected or shotgun) have been suggested as a means of improving efficiency of ribozymes for gene suppression (Ohkawa et al., Nucleic Acids Symp Ser. (29):121-2, 1993).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for future suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). In some cases suppression strategies have led to a reduction in RNA levels without a concomitant reduction in proteins, whereas in others, reductions in RNA have been mirrored by reductions in protein.

The diverse array of suppression strategies that can be employed includes the use of DNA and/or RNA aptamers that can be selected to target, for example, a protein of interest such as an RESE. For example, in the case of age related macular degeneration (AMD), anti-VEGF aptamers have been generated and have been shown to provide clinical benefit in some AMD patients (Ulrich H, et al. Comb. Chem. High Throughput Screen 9: 619-632, 2006). Suppression and replacement using aptamers for suppression in conjunction with a modified replacement gene and encoded protein that is refractory or partially refractory to aptamer-based suppression could be used in the invention.

In one embodiment, a method for identifying compounds or compositions that inhibit RAS-mediated tumor formation or growth comprising contacting a cell with a compound or composition and assaying for decreased expression of one or more RESEs. The screening may be carried out in vitro or in vivo using any of the experimental frameworks disclosed herein, or any experimental framework known to one of ordinary skill in the art to be suitable for contacting cells with a compound or composition and assaying for alterations in the expression of one or more RESEs.

In one aspect compounds are contacted with test cells (and preferably control cells) at a predetermined dose. In one embodiment the dose may be about up to 1 nM. In another embodiment the dose may be between about 1 nM and about 100 nM. In another embodiment the dose may be between about 100 nM and about 10 uM. In another embodiment the dose may be at or above 10 uM. Following incubation for an appropriate predetermined time, the effect of compounds on the expression of the one or more Ras epigenetic silencing effectors (RESE) is determined by an appropriate method known to one of ordinary skill in the art. In one embodiment, quantitative RT-PCR is employed to examine the expression of RESEs. Other methods known to one of ordinary skill in the art could be employed to analyze mRNA levels, for example microarray analysis, cDNA analysis, Northern analysis, and RNase Protection Assays. Compounds that substantially alter the expression of one or more metastasis suppressors genes can be used for treatment and/or can be examined further.

In other embodiments, expression of RESEs is assessed by examining protein levels, by an appropriate method known to one of ordinary skill in the art, such as western analysis. Other methods known to one of ordinary skill in the art could be employed to analyze proteins levels, for example immunohistochemistry, immunocytochemistry, ELISA, Radioimmunoassays, proteomics methods, such as mass spectroscopy or antibody arrays.

Still other parameters disclosed herein that are relevant to Ras epigenetic silencing could provide a basis for screening for compounds. In one embodiment, the epigenetic state (e.g., degree of CpG methylation) at a DNA regulatory region of a Ras responsive gene (e.g., Fas) could be assayed in a compound screen. For example, the methylated DNA immunoprecipitation (MeDIP) assay described herein could be used to assay the epigenetic state at the DNA regulatory region. The cellular location of a RESE could also be assessed. For example, the binding of an RESE to the DNA regulatory region of a Ras responsive gene (e.g., Fas) could be assayed in a compound screen. In one embodiment, the assay comprises an expression construct that includes a DNA regulatory region of the Ras responsive gene and that encodes a reporter gene product (e.g., a luciferase enzyme), wherein expression of the reporter gene is correlated with the binding of an RESE to the included DNA regulatory region. In this embodiment assessment of reporter gene expression (e.g., luciferase activity) provides an indirect method for assessing the binding of an RESE to the DNA regulatory region of a Ras responsive gene. This and other similar assays will be well known to one of ordinary skill in the art. In other embodiments, Chromatin immunoprecipitation assays could be used to assess the binding of a RESE with a regulatory DNA region of a Ras responsive gene.

As described above, compounds or compositions that substantially alter the expression of one or more RESEs and/or that are potential modulators of Ras dependent tumor growth can be discovered using the disclosed test methods. Examples of types of compounds or compositions that may be tested include, but are not limited to: anti-metastatic agents, cytotoxic agents, cytostatic agents, cytokine agents, anti-proliferative agents, immunotoxin agents, gene therapy agents, angiostatic agents, cell targeting agents, etc.

The following provides further examples of test compounds and is not meant to be limiting. Those of ordinary skill in the art will recognize that there are numerous additional types of suitable test compounds that may be tested using the methods, cells, and/or animal models of the invention. Test compounds can be small molecules (e.g., compounds that are members of a small molecule chemical library). The compounds can be small organic or inorganic molecules of molecular weight below about 3,000 Daltons. The small molecules can be, e.g., from at least about 100 Da to about 3,000 Da (e.g., between about 100 to about 3,000 Da, about 100 to about 2,500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da). Test compounds can also be microorganisms, such as bacteria (e.g., Escherichia coli, Salmonella typhimurium, Mycobacterium avium, or Bordetella pertussis), fungi, and protists (e.g., Leishmania amazonensis), which may or may not be genetically modified. See, e.g., U.S. Pat. Nos. 6,190,657 and 6,685,935 and U.S. Patent Applications No. 2005/0036987 and 2005/0026866.

The small molecules can be natural products, synthetic products, or members of a combinatorial chemistry library. A set of diverse molecules can be used to cover a variety of functions such as charge, aromaticity, hydrogen bonding, flexibility, size, length of side chain, hydrophobicity, and rigidity. Combinatorial techniques suitable for synthesizing small molecules are known in the art (e.g., as exemplified by Obrecht and Villalgrodo, Solid-Supported Combinatorial and Parallel Synthesis of Small-Molecular-Weight Compound Libraries, Pergamon-Elsevier Science Limited (1998)), and include those such as the “split and pool” or “parallel” synthesis techniques, solid-phase and solution-phase techniques, and encoding techniques (see, for example, Czamik, A. W., Curr. Opin. Chem. Biol. (1997) 1:60). In addition, a number of small molecule libraries are publicly or commercially available (e.g., through Sigma-Aldrich, TimTec (Newark, Del.), Stanford School of Medicine High-Throughput Bioscience Center (HTBC), and ChemBridge Corporation (San Diego, Calif.).

Compound libraries screened using the new methods can comprise a variety of types of test compounds. A given library can comprise a set of structurally related or unrelated test compounds. In some embodiments, the test compounds are peptide or peptidomimetic molecules. In some embodiments, test compounds include, but are not limited to, peptide analogs including peptides comprising non-naturally occurring amino acids, phosphorous analogs of amino acids, amino acids having non-peptide linkages, or other small organic molecules. In some embodiments, the test compounds are peptidomimetics (e.g., peptoid oligomers, e.g., peptoid amide or ester analogues, D-peptides, L-peptides, oligourea or oligocarbamate); peptides (e.g., tripeptides, tetrapeptides, pentapeptides, hexapeptides, heptapeptides, octapeptides, nonapeptides, decapeptides, or larger, e.g., 20-mers or more); cyclic peptides; other non-natural peptide-like structures; and inorganic molecules (e.g., heterocyclic ring molecules). Test compounds can also be nucleic acids.

The test compounds and libraries thereof can be obtained by systematically altering the structure of a first “hit” compound that has a chemotherapeutic (e.g., anti-RESE) effect, and correlating that structure to a resulting biological activity (e.g., a structure-activity relationship study).

Such libraries can be obtained using any of the numerous approaches in combinatorial library methods known in the art, including: peptoid libraries (libraries of molecules having the functionalities of peptides, but with a novel, non-peptide backbone which are resistant to enzymatic degradation but which nevertheless remain bioactive; see, e.g., Zuckermann, et al., J. Med. Chem., 37:2678-85 (1994)); spatially addressable parallel solid phase or solution phase libraries; synthetic library methods requiring deconvolution; the “one-bead one-compound” library method; and synthetic library methods using affinity chromatography selection (Lam, Anticancer Drug Des. 12:145 (1997)). Examples of methods for the synthesis of molecular libraries can be found in the art, for example in: DeWitt et al., Proc. Natl. Acad. Sci. USA, 90:6909 (1993); Erb et al., Proc. Natl. Acad. Sci. USA, 91:11422 (1994); Zuckermann et al., J. Med. Chem., 37:2678 (1994); Cho et al., Science, 261:1303 (1993); Carrell et al., Angew. Chem. Int. Ed. Engl., 33:2059 (1994); Carell et al., Angew. Chem. Int. Ed. Engl., 33:2061 (1994); and in Gallop et al., J. Med. Chem., 37:1233 (1994). Libraries of compounds can be presented in solution (e.g., Houghten (1992) Biotechniques, 13:412-421), or on beads (Lam (1991) Nature, 354:82-84), chips (Fodor (1993) Nature, 364:555-556), bacteria (Ladner, U.S. Pat. No. 5,223,409), spores (Ladner, U.S. Pat. No. 5,223,409), plasmids (Cull et al. (1992) Proc. Natl. Acad. Sci. USA, 89:1865-1869) or on phage (Scott and Smith (1990) Science, 249:386-390; Devlin (1990) Science, 249:404-406; Cwirla et al. (1990) Proc. Natl. Acad. Sci. USA, 87:6378-6382; Felici (1991) J. Mol. Biol., 222:301-310; Ladner, supra.).

Certain results of the compound identification and characterization methods disclosed herein may be clinically beneficial, such as if the compound is a suppressor of Ras-dependent tumor growth and/or a suppressor of RESEs, such as those disclosed herein (See Table 1 and 2). Still other clinically beneficial results include: (a) inhibition or arrest of primary tumor growth, (b) inhibition of metastatic tumor growth and (c) extension of survival of a test subject. Compounds with clinically beneficial results are potential chemotherapeutics, and may be formulated as such.

Compounds identified as having a chemotherapeutic or anti-RESE effect can be selected and systematically altered, e.g., using rational design, to optimize binding affinity, avidity, specificity, or other parameters. Such optimization can also be screened for using the methods described herein. Thus, one can screen a first library of small molecules using the methods described herein, identify one or more compounds that are “hits,” (by virtue of, for example, induction of expression of one or more RESEs and/or their ability to reduce the size and/or number of Ras dependent tumors, e.g., at the original site of implantation and at metastasis sites), and subject those hits to systematic structural alteration to create a second library of compounds structurally related to the hit. The second library can then be screened using the methods described herein.

A variety of techniques useful for determining the structures of compounds are known and can be used in the methods described herein (e.g., NMR, mass spectrometry, gas chromatography equipped with electron capture detectors, fluorescence, and absorption spectroscopy).

Assays of chemotherapeutic activity of test compounds may be conducted in vitro or ex vivo and/or in vivo using cells (e.g., Ras-transformed cells) and methods of the invention. For example, a test compound may be administered to a nonhuman subject to which has been administered (e.g., implanted or injected with) a plurality of the cells (e.g., Ras-transformed cells) described herein, e.g., a number of Ras-transformed cells sufficient to induce the formation of one or more tumors (e.g., Ras-dependent tumors). The nonhuman subject can be, e.g., a rodent (e.g., a mouse). The test compound can be administered to the subject by any regimen known in the art. For example, the test compound can be administered prior to, concomitant with, and/or following the administration of Ras-transformed cells of the invention. A test compound can also be administered regularly throughout the course of the method, for example, one, two, three, four, or more times a day, weekly, bi-weekly, or monthly, beginning before or after cells of the invention have been administered. In other embodiments, the test compound is administered continuously to the subject (e.g., intravenously). The dose of the test compound to be administered can depend on multiple factors, including the type of compound, weight of the subject, frequency of administration, etc. Determination of dosages is routine for one of ordinary skill in the art. Typical dosages are 0.01-200 mg/kg (e.g., 0.1-20 or 1-10 mg/kg).

The size and/or number of tumors (e.g., Ras-dependent tumors) in the subject can be determined following administration of the tumor cells and the test compound. The size and/or number of tumors can be determined non-invasively by any means known in the art. For example, tumor cells that are fluorescently labeled (e.g., by expressing a fluorescent protein such as GFP) can be monitored by various tumor-imaging techniques or instruments, e.g., non-invasive fluorescence methods such as two-photon microscopy. The size of a tumor implanted subcutaneously can be monitored and measured underneath the skin.

To determine whether a compound affects Ras-dependent tumor formation or the growth of Ras-transformed cells, the size and/or number of tumors in the subject can be compared to a reference standard (e.g., a control value). A reference standard can be a control subject which has been given the same regimen of administration of tumor cells and test compound, except that the test compound is omitted or administered in an inactive form. Alternately, a compound believed to be inert in the system can be administered. A reference standard can also be a control subject which has been administered non-Ras-transformed cells and test compound, non-Ras-transformed cells and no test compound, or non-Ras-transformed cells and an inactive test compound. The reference standard can also be a numerical figure or figures representing the size and/or number of Ras-dependent tumors expected in an untreated subject. This numerical figure(s) can be determined by observation of a representative sample of untreated subjects. A reference standard may also be the test animal before administration of the compound.

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The PolymeRase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### EXAMPLES

### Example 1

### RNAi Screen

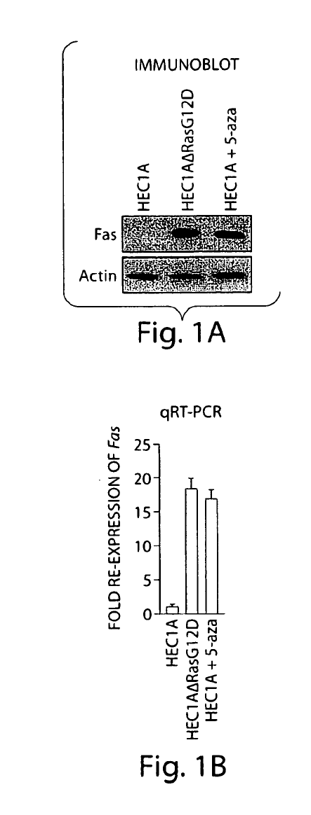

Members of the ras oncogene family transform most immortalized cell lines, and mutations of ras genes occur in ˜30% of human tumours (Giehl, K, Biol. Chem. 386, 193-205 (2005)). In addition, activation of the Ras pathway is frequent in human tumours even in the absence of ras mutations (Ehmann, F. et al., Leuk. Lymphoma 47, 1387-1391 (2006)). Previous studies have shown that in mouse NIH 3T3 cells activated Ras epigenetically silences Fas expression thereby preventing Fas-ligand induced apoptosis (Fenton, R. G., Hixon, J. A., Wright, P. W., Brooks, A. D. & Sayers, T. J., Cancer Res. 58, 3391-3400 (1998); Peli, J. et al., EMBO J. 18, 1824-1831 (1999)). Activated Ras also epigenetically silences Fas expression in the human K-ras transformed cell line, HEC1A (FIG. 1). In addition, epigenetic silencing of Fas occurs in some transformed cells, human tumours, and mouse models of cancer, and this silencing is relevant to tumour progression (Hopkins-Donaldson, S. et al., Cell Death Differ. 10, 356-364 (2003)).

To identify genes required for Ras-mediated silencing of Fas, we performed a genome-wide small hairpin RNA (shRNA) screen using, as a selection strategy, re-expression of Fas protein on the cell surface (FIG. 2a). A mouse shRNA library comprising ˜62,400 shRNAs directed against ˜28,000 genes was divided into 10 pools, which were packaged into retrovirus particles and used to stably transduce Fas-negative, K-ras NIH 3T3 cells. Fas-positive cells in each pool were selected on immunomagnetic beads using an anti-Fas antibody, the Fas-positive population was expanded, and the shRNAs were identified by sequence analysis. Positive candidates were confirmed by stably transducing K-ras NIH 3T3 cells with single shRNAs directed against the candidate genes followed by immunoblot analysis for Fas re-expression.

### Example 2

### Hit Identification and Validation

### Ras epigenetic silencing effectors (RESEs)

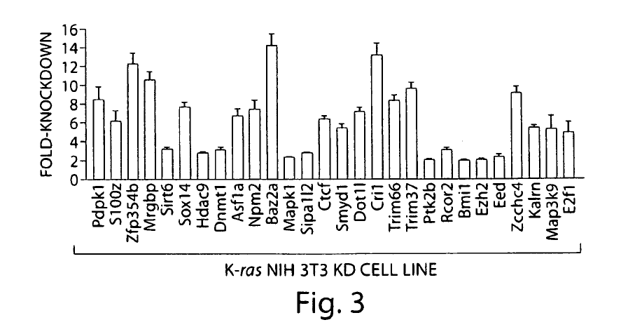

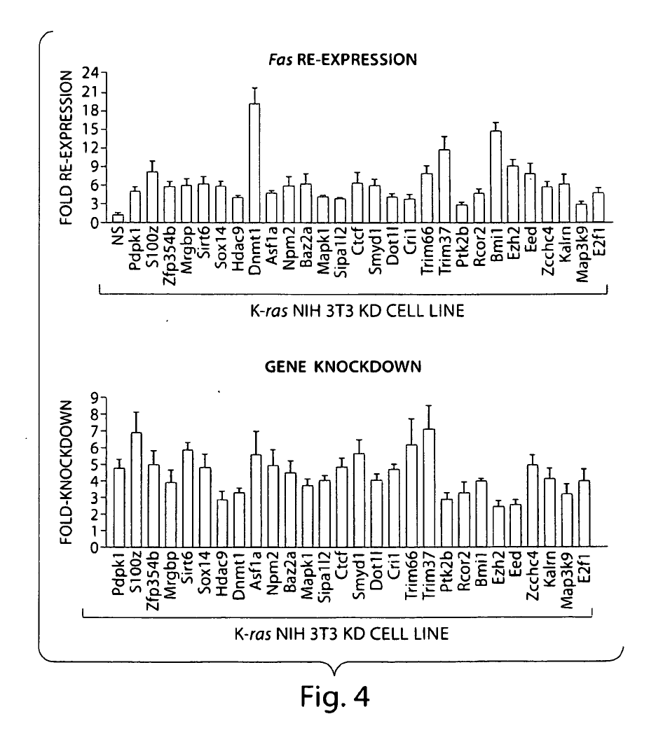

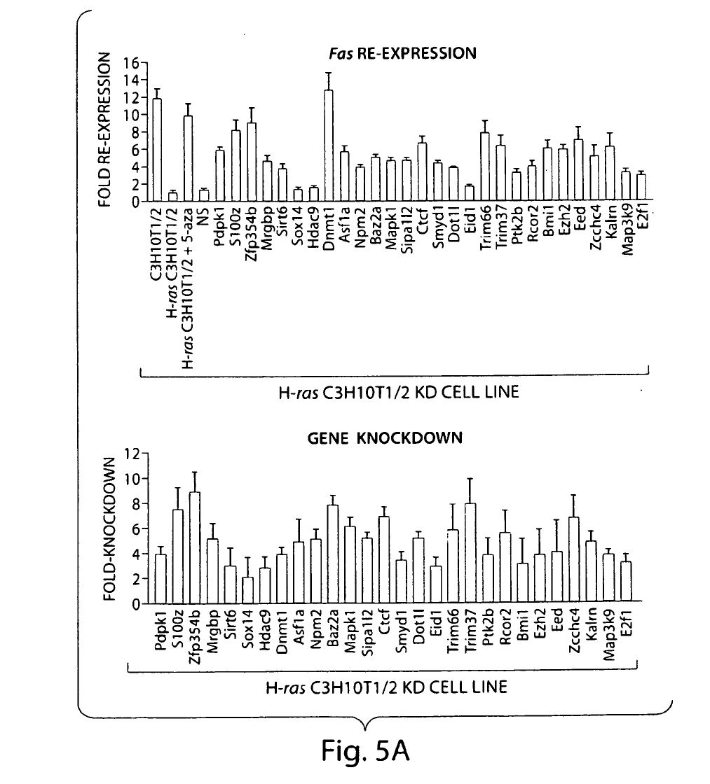

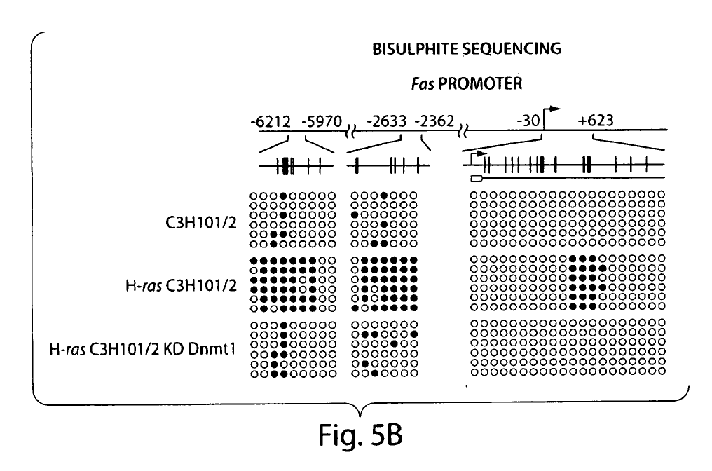

The screen identified 28 genes that, following shRNA-mediated knockdown, resulted in Fas re-expression. These genes are listed in Tables 1 and 2 and immunoblot analysis of Fas re-expression in the 28 K-ras NIH 3T3 knockdown (K-ras NIH 3T3 KD) cell lines is shown in FIG. 2b. Consistent with previous reports (Peli, J. et al., EMBO J. 18, 1824-1831 (1999)), treatment of K-ras NIH 3T3 cells with the DNA methylation inhibitor 5-aza-2′-deoxycytidine (5-aza) restored Fas expression (see also FIG. 1). Quantitative real-time RT-PCR (qRT-PCR) confirmed in all cases that expression of the target gene was decreased in each K-ras NIH 3T3 KD cell line (FIG. 3). For all 28 genes, a second, unrelated shRNA directed against the same target also resulted in Fas re-expression when stably expressed in K-ras NIH 3T3 cells (FIG. 4). Knockdown of each of these 28 genes in an additional cell line, H-ras transformed murine C3H101/2 cells, also derepressed the epigenetically silenced Fas gene (FIG. 5).

For convenience, we will refer to the protein products of the 28 genes as Ras epigenetic silencing effectors (RESEs). The RESEs include cytoplasmic cell signalling molecules and nuclear regulators of gene expression (Tables 1 and 2). Among the cell signalling components, PDPK1, a serine-threonine kinase, is known to function downstream of Ras and to regulate the PI3K-AKT pathway, which is frequently activated in cancer (Osaki, M., Oshimura, M. & Ito, H., Apoptosis 9, 667-676 (2004)). Significantly, it has been previously reported that the PI3K-AKT pathway is involved in Ras-mediated silencing of Fas (Peli, J. et al., EMBO J. 18, 1824-1831 (1999)). Other cell signalling proteins include two members of the MAP kinase family (MAP3K9/MLK1 and MAPK1/ERK2), a tyrosine kinase (PTK2B), a RhoGEF kinase (KALRN), and a calcium-binding regulatory protein (S100Z). Notably, MAPK1 is a proximal Ras target that is frequently activated in cancer (de Vries-Smits, A. M., Burgering, B. M., Leevers, S. J., Marshall, C. J. & Bos, J. L., Nature 357, 602-604 (1992)), and PTK2B is recruited to cell membranes by activated Ras (Alfonso, P. et al., Proteomics 6 Suppl 1, S262-271 (2006)).

Among the nuclear gene regulatory proteins are known transcriptional activators and repressors/corepressors (CTCF, EID1, E2F1, RCOR2, and TRIM66/TIF1D) including a number of Polycomb group proteins (BMI1, EED, and EZH2); several predicted sequence-specific DNA binding proteins (SOX14, ZCCHC4, and ZFP345B); three histone methyltransferases (DOT1L, EZH2, and SMYD1); a histone deacetylase (HDCA9); two histone chaperones (ASF1A and NPM2); and the maintenance DNA methyltransferase DNMT1. Significantly, many of the nuclear RESEs are involved in chromatin modification, a process closely associated with DNA methylation (Klose, R. J. & Bird, A. P., Trends Biochem. Sci. 31, 89-97 (2006)). Surprisingly, one of the nuclear RESEs is BAZ2A/TIP5, previously known only to be involved in repression of RNA polymerase I-directed ribosomal gene transcription (Zhou, Y., Santoro, R. & Grummt, I., EMBO J. 21, 4632-4640 (2002)).

### Example 3

### RESE Expression Analysis

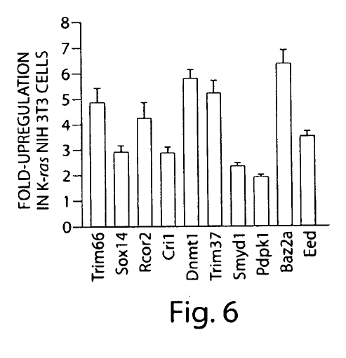

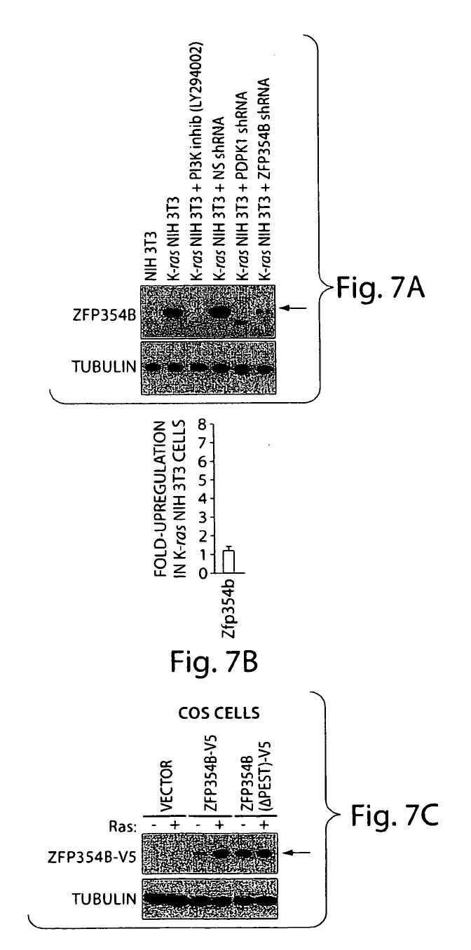

A number of RESEs were substantially upregulated at the transcriptional (FIG. 6) or post-transcriptional (FIG. 7) level in K-ras NIH 3T3 cells compared to NIH 3T3 cells, explaining, at least in part, how K-ras activates this silencing pathway. One of the genes we found transcriptionally upregulated in K-ras NIH 3T3 cells was Dnmt1 (FIG. 6); consistent with our results, it has been previously reported that Dnmt1 is upregulated in K-ras transformed rat intestinal epithelial (RIE-1) cells (Pruitt, K. et al., J. Biol. Chem. 280, 23363-23370 (2005)) and in oncogenic Ha-ras-transfected adrenocortical tumour cells (MacLeod, A. R., Rouleau, J. & Szyf, M., J. Biol. Chem. 270, 11327-11337 (1995)).

### Example 4

### Epigenetic Assessment of the Fas Gene

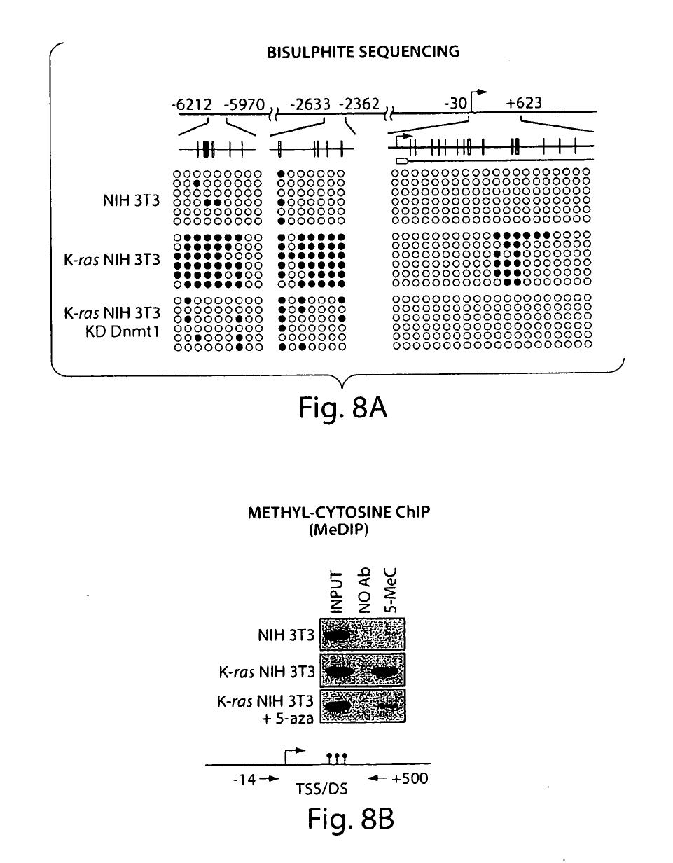

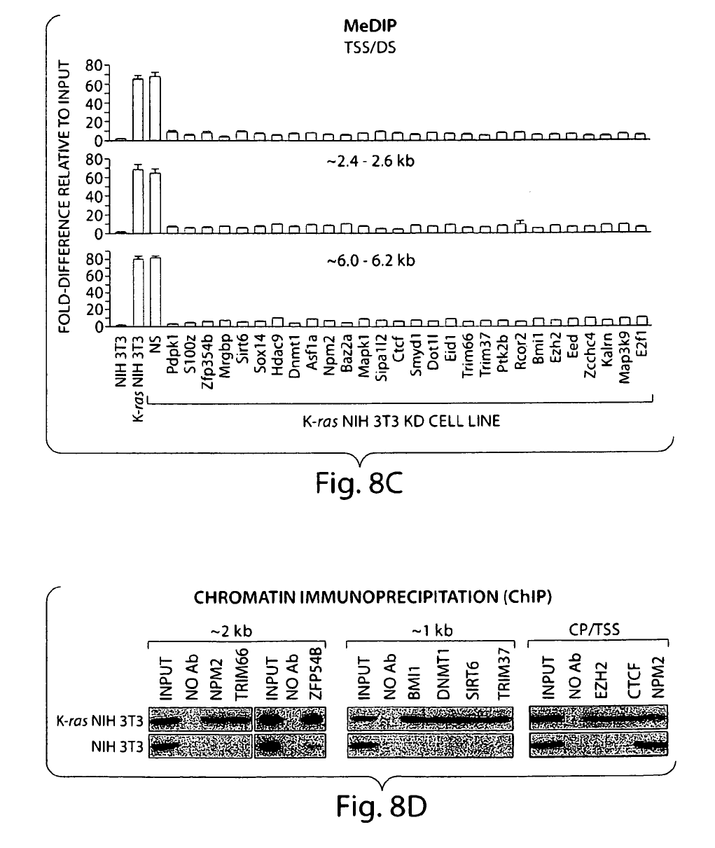

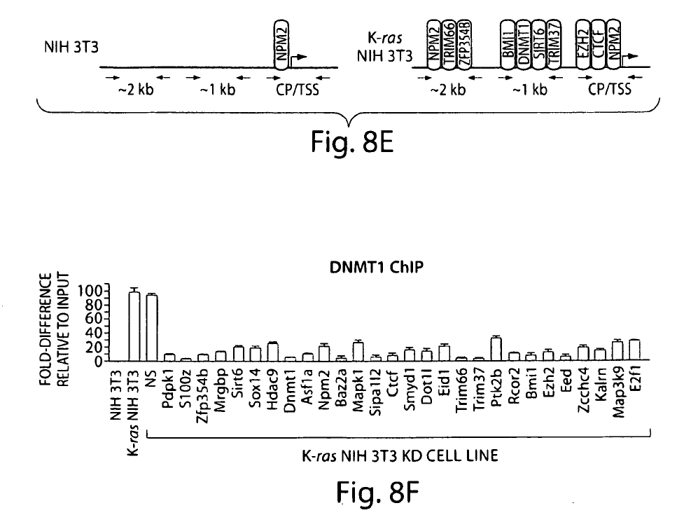

As mentioned above, treatment of K-ras NIH 3T3 cells with 5-aza results in Fas re-expression, suggesting that repression is due, at least in part, to promoter hypermethylation. We therefore sought to determine the relationship between Fas promoter hypermethylation and Fas re-expression following knockdown of each of the 28 RESEs. We first confirmed that the repressed Fas promoter was hypermethylated and mapped the hypermethylated region(s) by bisulphite sequence analysis. These results, summarized in FIG. 8a, reveal three regions located upstream and downstream from the transcription start-site (TSS) that are hypermethylated in K-ras NIH 3T3 cells but not in NIH 3T3 cells or in K-ras NIH-3T3 cells following knockdown of DNMT1. Significantly, these same three Fas promoter regions are also hypermethylated in H-ras transformed C3H10T1/2 cells but not in C3H10T1/2 cells or in H-ras transformed C3H10T1/2 cells following knockdown of DNMT1 (FIG. 5b).

To facilitate analysis of the methylation status of these three regions in the 28 K-ras NIH 3T3 KD cell lines we established and validated a rapid methylated DNA immunoprecipitation (MeDIP) assay, in which the antibody is directed against 5-methyl-cytosine (Weber, M. et al., Nat. Genet. 37, 853-862 (2005)). The MeDIP results of FIG. 8b show that in K-ras NIH 3T3 cells the Fas promoter was hypermethylated within the TSS/downstream (DS) region consistent with the bisulphite sequencing results. Moreover, the MeDIP results show, as expected, that the TSS/DS region was not hypermethylated in NIH 3T3 cells or in K-ras NIH 3T3 cells following 5-aza treatment. We then assessed the three hypermethylated Fas promoter regions in each of the 28 K-ras NIH 3T3 KD cell lines. The results of FIG. 8c show that in all 28 K-ras NIH 3T3 KD cell lines the three Fas promoter regions were not hypermethylated, consistent with the expression data.

### Example 5

### Assessment of RESE Binding to Regulatory Regions of the Fas gene

To further understand the basis of Fas silencing, we asked whether nuclear RESEs functioned by direct association with the Fas promoter. We performed a series of chromatin immunoprecipitation (ChIP) assays, based upon antibody availability, using three sets of promoter-specific primer-pairs located ˜2 kb upstream of the TSS, ˜1 kb upstream of the to TSS or encompassing the core promoter/TSS. The three primer-pairs cover the entire Fas promoter region. FIG. 8d shows that in K-ras NIH 3T3 cells, nine of the RESEs were bound to specific Fas promoter regions: NPM2, TRIM66 and ZFP354B were present ˜2 kb upstream of the TSS; BMI1, DNMT1, SIRT6 and TRIM37 were present ˜1 kb upstream of the TSS; and EZH2, CTCF and NPM2 were present at the core promoter/TSS. Significantly, in NIH 3T3 cells only NPM2 was detectably associated with the Fas promoter at the core promoter/TSS. Thus, as summarized in FIG. 8e, at least nine RESEs are recruited to specific regions of the Fas promoter in response to expression of activated Ras.

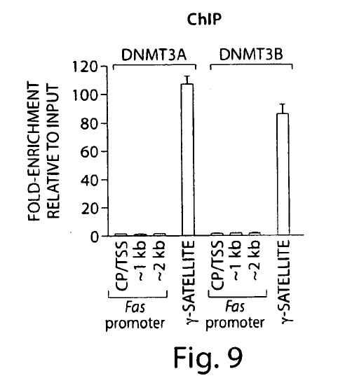

The ChIP results of FIG. 8d show that DNMT1 is associated with the Fas promoter in K-ras NIH 3T3 cells but not in untransformed NIH 3T3 cells. The two other DNA methyltransferases, DNMT3A and DNMT3B, were not identified in the original shRNA screen and are not detectably associated with the Fas promoter by ChIP analysis (FIG. 9). These results strongly suggest that DNMT1 is required to sustain hypermethylation of the Fas promoter in K-ras NIH 3T3 cells. To confirm this possibility, we analyzed association of DNMT1 with the Fas promoter in the 28 K-ras NIH 3T3 KD cell lines. The ChIP results of FIG. 8f show that in all 28 K-ras NTH 3T3 KD cell lines association of DNMT1 with the Fas promoter was markedly reduced. Moreover, bisulphite sequence analysis showed that following knockdown of DNMT1 the TSS/DS region of the Fas promoter was no longer hypermethylated (FIG. 8a). Collectively, these results indicate that RNAi-mediated knockdown of any of the 28 RESEs results in a failure to recruit DNMT1 to the Fas promoter, loss of Fas promoter hypermethylation and de-repression of Fas expression.

### Example 6

### Assessment of DNA Methylation and RESE Binding at Regulatory Regions of Ras-Regulated Genes

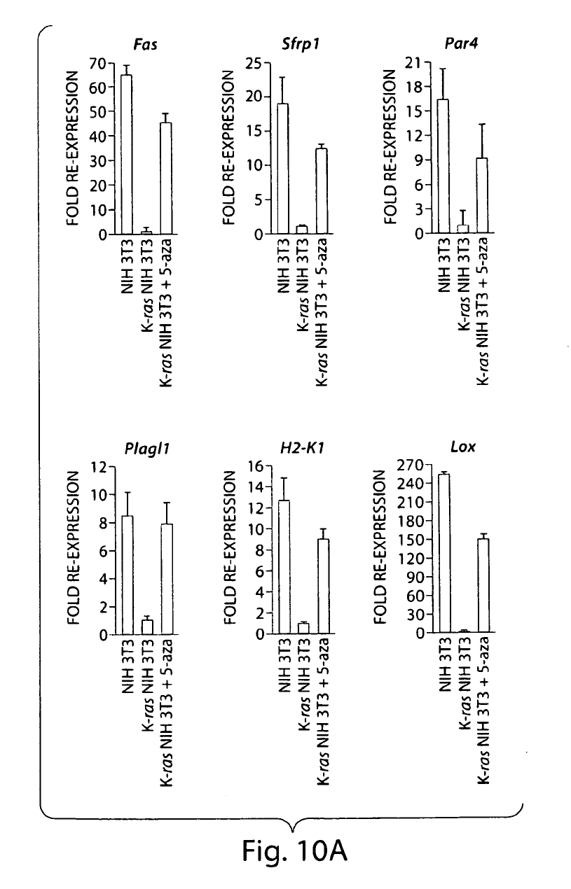

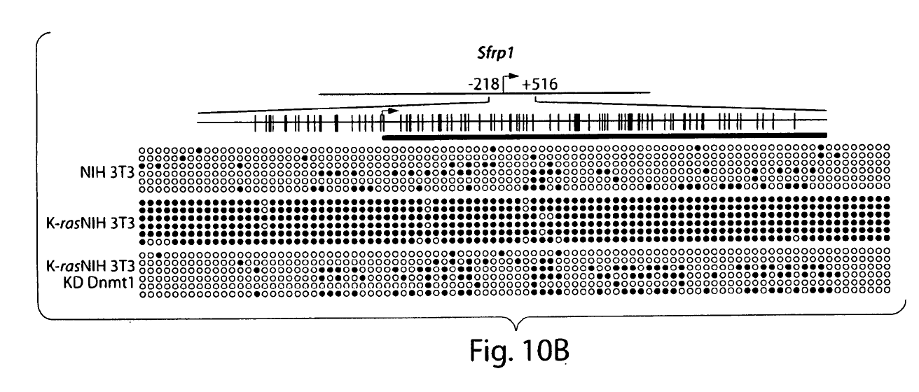

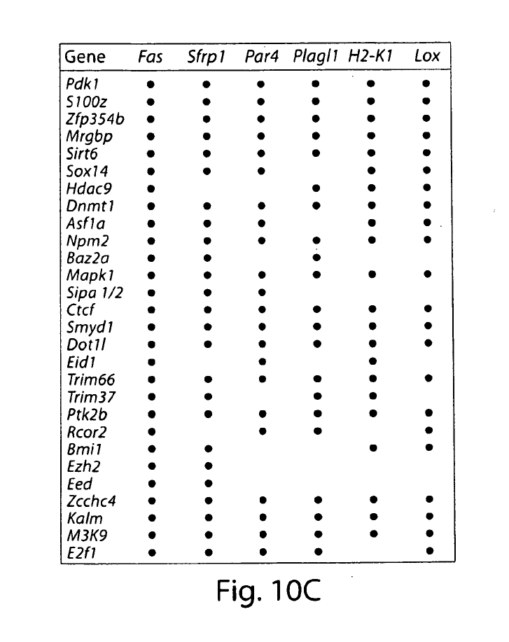

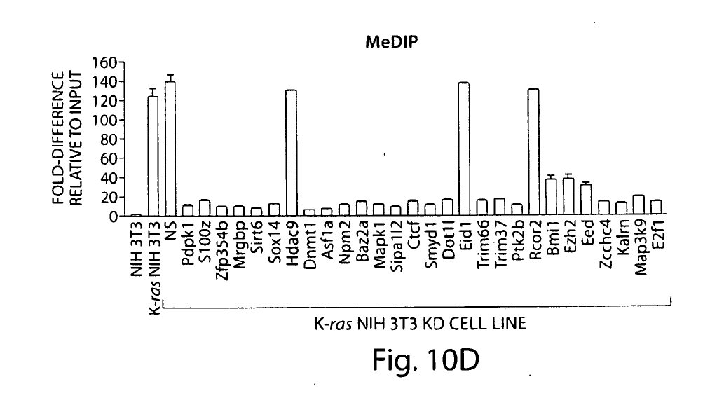

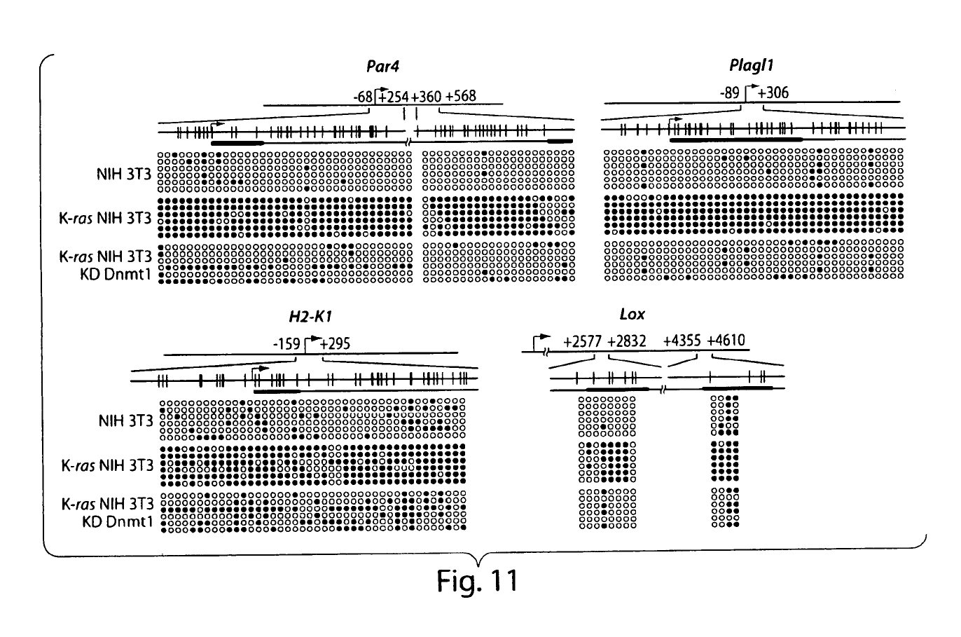

A number of genes in addition to Fas are known to be epigenetically silenced in ras transformed cells. To gain insight into whether Ras mediates epigenetic silencing of different genes through common or diverse pathways, we analyzed five other well-studied, epigenetically silenced genes: Sfrp1, Par4/Pawr, Plagl1, H2-K1 and Lox. A variety of evidence supports the relevance of these genes to cellular transformation and cancer (reviewed in (Ranganathan, P. & Rangnekar, V. M. Ann. N Y Acad. Sci. 1059, 76-85 (2005); Kenyon, K. et al. Science 253, 802 (1991); Nie, Y. et al. Carcinogenesis 22, 1615-1623 (2001); Abdollahi, A. J. Cell Physiol. 210, 16-25 (2007); Rubin, J. S., Barshishat-Kupper, M., Feroze-Merzoug, F. & Xi, Z. F. Front. Biosci. 11, 2093-2105 (2006))). The results of FIG. 10a show that, like Fas, all five genes were expressed in NIH 3T3 cells but not in K-ras NIH 3T3 cells, and were re-expressed in K-ras NIH 3T3 cells following treatment with 5-aza. Bisulphite sequence analysis confirmed that all five genes contained regions that are hypermethylated in K-ras NIH3T3 cells but not in NIH3T3 cells or in K-ras NIH3T3 cells following knockdown of DNMT1 (FIGS. 10b and 11a). For four of these genes (Sfrp1, Par4, Plag1 and H2-K1), the TSS was encompassed by densehypermethylation in K-ras NIH 3T3 cells.

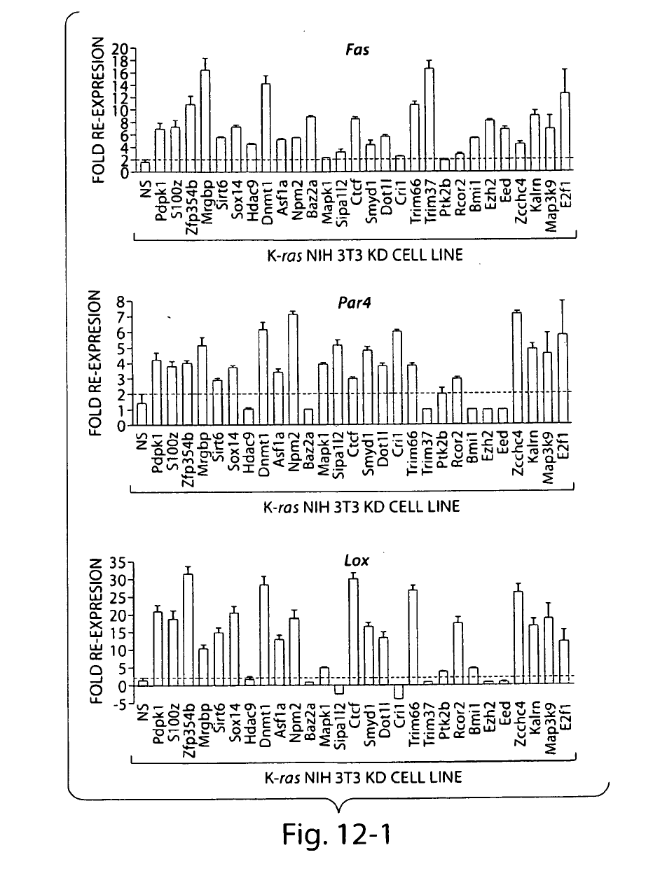

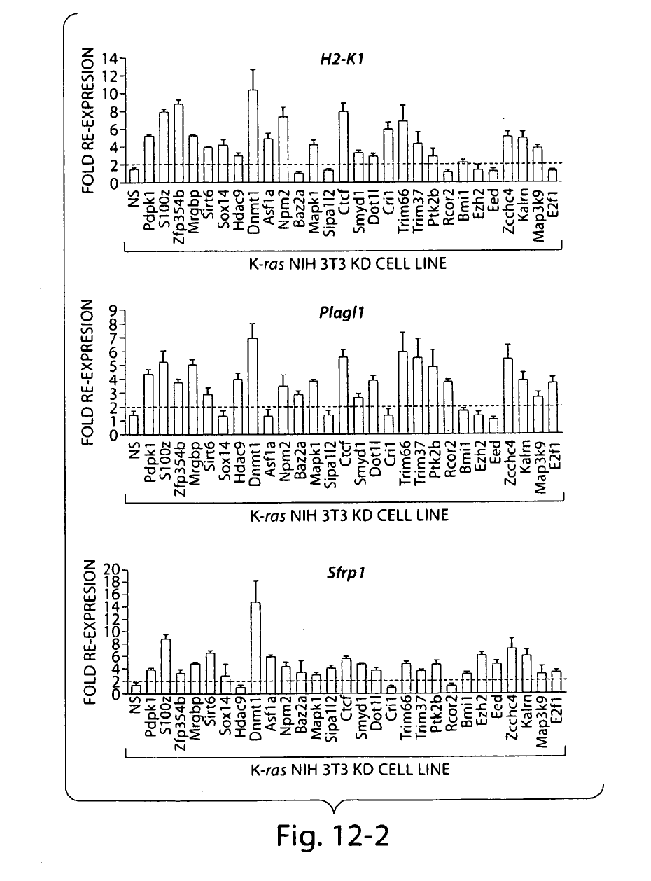

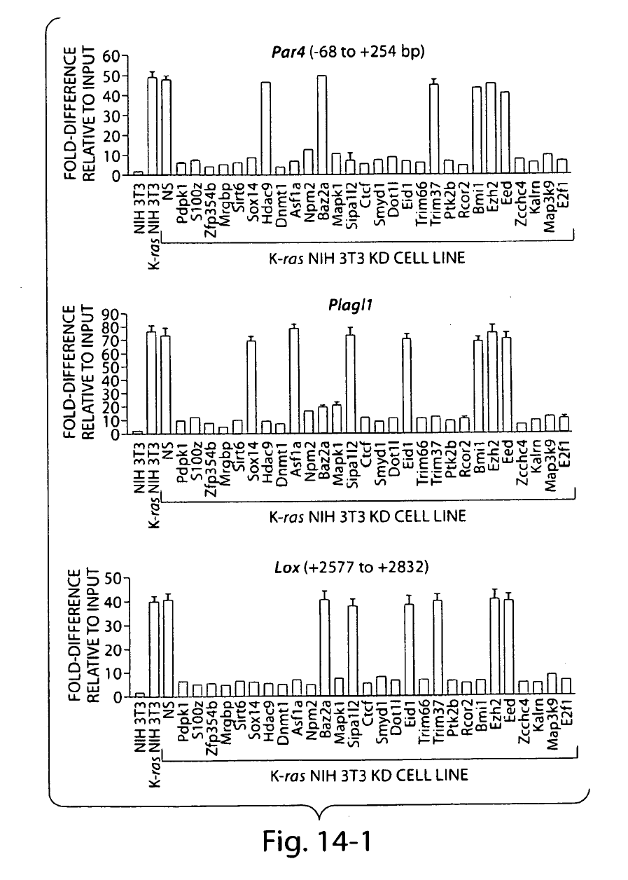

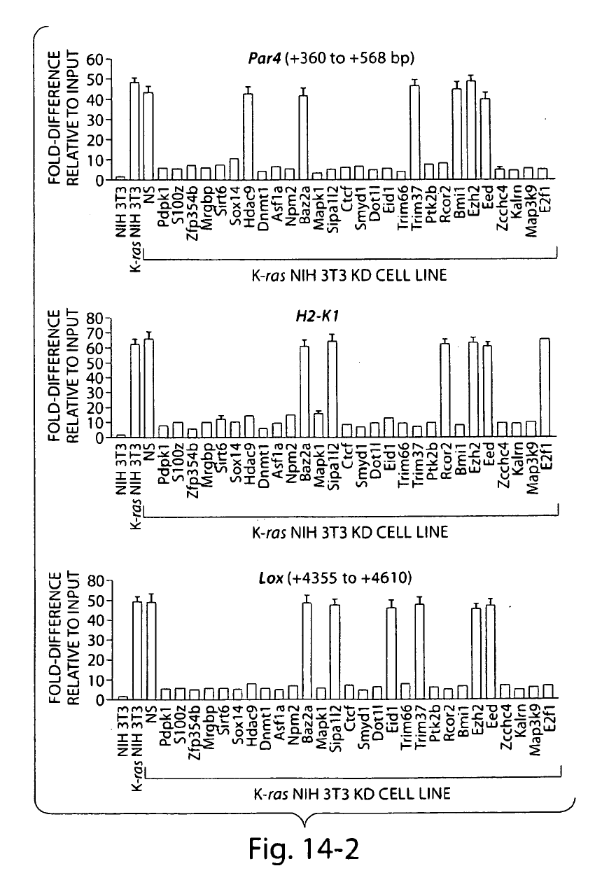

We next analyzed expression of Sfrp1, Par4, Plagl1, H2-K1, and Lox in the 28 K-ras NIH 3T3 KD cell lines. The qRT-PCR results of FIG. 12 are summarized in FIG. 10c and reveal substantial overlap in the requirements of RESEs for epigenetic silencing of Fas, Sfrp1, Par4, Plagl1, H2-K1, and Lox: of the 28 RESEs required for silencing of Fas, at least 21 were also required for silencing of each of the five other genes analyzed. MeDIP analysis for all five gene genes revealed a perfect correspondence between the RESEs required for silencing and for promoter hypermethylation (FIGS. 10d and 14). These results indicate that Ras directs the epigenetic silencing of multiple, unrelated genes through a largely common pathway.

### Example 7

### Involvement of RESEs in Ras-Mediated Transformation

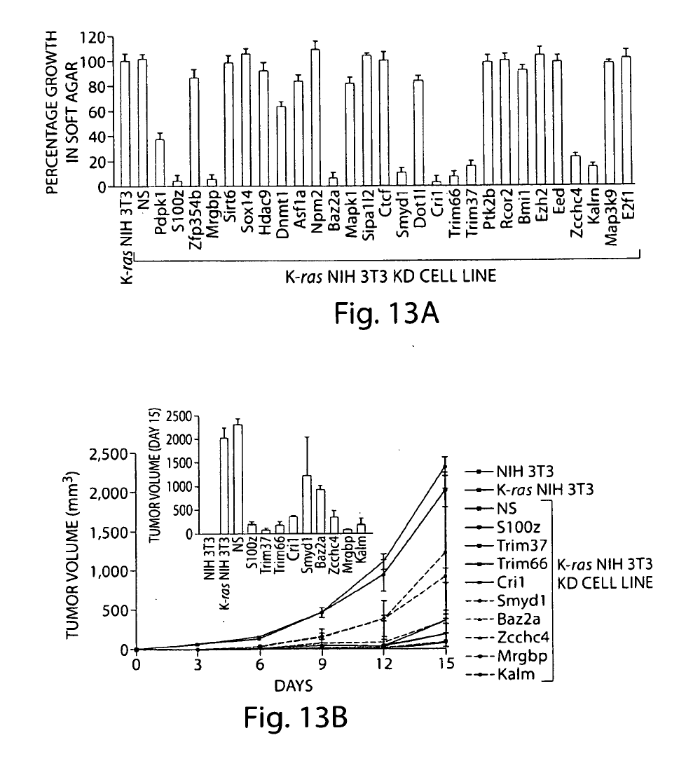

Proteins that function downstream of Ras could be essential for a fully transformed phenotype. To determine whether any of the 28 RESEs were also required for Ras-mediated transformation, we first tested the ability of the K-ras NTH 3T3 KD cell lines to grow in soft agar. FIG. 13a shows that knockdown of any of nine RESEs (S100Z, MRGBP, BAZ2A, SMYD1, EID1, TRIM66, TRIM37, ZCCHC4, and KALRN) markedly inhibited anchorage-independent growth.

### Example 8

### Involvement of RESEs in Ras-Mediated Tumor Growth

To further characterize the role of these nine RESEs in Ras-mediated transformation, we tested the ability of the corresponding nine K-ras NIH 3T3 KD cell lines to form tumours following subcutaneous injection in the flanks of nude mice. The results of FIG. 13b show that knockdown of SMYD1 or BAZ2A moderately inhibited tumour growth, whereas knockdown of S100Z, TRIM37, TRIM66, EID1, ZCCHC4, MRGBP, or KALRN markedly inhibited tumour growth.

It is well established that in many cancers specific genes affecting cellular growth control are hypermethylated and epigenetically silenced (Baylin, S. B., Nat. Clin. Pract. Oncol. 2, Suppl 1, S4-11 (2005); Esteller, M., Br. J. Cancer 94, 179-183 (2006)). However, the mechanistic basis of epigenetic silencing is not understood. According to one model, an epigenetic event, such as hypermethylation of a CpG-rich promoter region, occurs randomly and non-specifically and the resulting alteration in gene expression confers a selectable growth advantage (Jones, P. A., Cancer Res. 56, 2463-2467 (1996)). In a second model, epigenetic silencing occurs through a specific pathway, comprising a defined set of components, initiated by an oncogene (Baylin, S. & Bestor, T. H., Cancer Cell 1, 299-305 (2002); Keshet, I. et al., Nat. Genet. 38, 149-153 (2006).

The results presented here demonstrate that oncogenic Ras directs epigenetic silencing through a specific unexpectedly complex pathway. We have shown that Ras-mediated epigenetic silencing requires at least 28 components (RESEs) that when knocked down, leads to Fas re-expression in K-ras NIH 3T3 cells. The large number of RESEs, which function non-redundantly, was surprising. A striking example of this unanticipated complexity and non-redundancy is that Ras-mediated silencing of Fas requires multiple transcriptional repressors/corepressors (CTCF, RCOR2, EID1, and TRIM66/TIF 1D), histone methyltransferases (DOT1L, EZH2, and SMYD1) and histone chaperones (ASF1A and NPM2.

Our ChIP analysis revealed that knockdown of any of the 28 RESEs resulted in a failure to recruit DNMT1 to the Fas promoter, loss of Fas promoter hypermethylation and de-repression of Fas expression. Our interpretation of these results is that assembly of an epigenetically repressed Fas promoter is a highly cooperative process that culminates in the recruitment of DNMT1. Consistent with this idea, BAZ2A and the Polycomb group protein EZH2, both of which were identified in this study as RESEs, are reported to physically associate with DNMT1 and may provide a platform for DNMT1 recruitment (Strohner, R. et al., EMBO J. 20, 4892-4900 (2001); Zhou, Y. & Grummt, I., Curr. Biol. 15, 1434-1438 (2005); Vire, E. et al., Nature 439, 871-874 (2006)).

The vast majority of RESEs have not been previously connected to the Ras pathway, and thus our results have identified a number of new factors that act downstream of Ras. More importantly, we found nine RESEs that are required for anchorage-independent growth and tumorigenicity; these nine factors represent novel downstream effectors of Ras required for transformation. Histone deacetylase inhibitors, which broadly and non-selectively interfere with epigenetic silencing, have proven to be beneficial anti-cancer agents (Yoo, C. B. & Jones, P. A., Nat. Rev. Drug Discov. 5, 37-50 (2006)). More efficacious therapeutics may be obtained by selectively inhibiting the epigenetic silencing pathway initiated by the oncogene. Thus, the identification of new components that act downstream of Ras, and are required for epigenetic silencing and complete transformation, provides potential new anti-cancer targets.

### METHODS

Cell culture. NIH 3T3 (ATCC# CRL-1658) and K:Molv NIH 3T3 (ATCC# CRL-6361; referred to here as K-ras NIH 3T3) cells were maintained in DMEM supplemented with 10% FCS at 37° C. and 5% CO₂. For 5-aza-2′-deoxycytidine (5-aza) treatment, K-ras NIH 3T3 cells were treated with 10 μM 5-aza for 72 h.

ShRNA screen. The mouse shRNA^{mir} library (release 2.16; Open Biosystems) was obtained through the University of Massachusetts Medical School shRNA library core facility. Ten retroviral pools, each comprising ˜6000 shRNA clones, were generated with titers of ˜2.6×10⁵pfu ml⁻¹. These retroviral stocks were produced following co-transfection into the PhoenixGP packaging cell line (a gift from G. Nolan, Stanford University, USA). K-ras NIH 3T3 cells (1.2×10⁶) were transduced at an MOI of 0.2 with the retroviral stocks in 100 mm plates, and 2 days later selected for resistance to puromycin (1.5 μg ml⁻¹) for 7 days. To isolate Fas-positive cells, 5×10⁶ cells from each pool were incubated with an anti-Fas antibody (15A7; eBiosciences) followed by incubation with IgG-conjugated magnetic beads (Miltenyi Biotec), and Fas-positive cells were selected using the Mini MACS magnetic separation system (Miltenyi Biotec) according to the manufacturer's instructions. The selected Fas-positive cells were expanded and genomic DNA isolated. To identify the candidate shRNAs, the shRNA region of the transduced virus was PCR amplified (using primers (SEQ ID NO: 1) PSM2-forward, 5′-GCTCGCTTCGGCAGCACATATAC-3′ and (SEQ ID NO: 2) PSM2-reverse, 5′-GAGACGTGCTACTTCCATTTGTC-3′) and cloned into pGEM-T Easy (Promega). An average of 30 clones were sequenced per pool (using primer (SEQ ID NO: 3) PSM2-seq, 5′-GAGGGCCTATTTCCCATGAT-3′). Individual to knockdown cell lines were generated by retroviral transduction of 0.6×10⁵ K-ras NIH 3T3 cells with the respective shRNA. Individual shRNAs were either obtained from the Open Biosystems library or synthesized (see Tables 3 and 4).

TABLE 3
Source ID numbers and clone locations for
shRNAs obtained from Open Biosystems

| Gene | Source ID | Clone Location |
|---|---|---|
| Asf1a | V2MM_64136 | SM2244-F-6 |
| | V2MM_71706 | SM2238-A-2 |
| Baz2a | V2MM_85159 | SM2467-F-2 |
| | V2MM_85157 | SM2108-H-8 |
| Bmi1 | V2MM_10594 | SM2169-C-12 |
| Eid1 | V2MM_61927 | SM2214-G-10 |
| | V2MM_70375 | SM2020-A-12 |
| Ctcf | V2MM_190309 | SM2165-B-1 |
| | V2MM_192417 | SM2165-D-3 |
| Dnmt1 | V2MM_46797 | SM2437-D-12 |
| Dot1l | V2MM_193454 | SM2256-A-8 |
| Eed | V2MM_73225 | SM2174-G-7 |
| | V2MM_65179 | SM2009-A-7 |
| Ezh2 | V2MM_30422 | SM2432-E-11 |
| | V2MM_35988 | SM2396-F-7 |
| E2f1 | V2MM_28115 | SM2433-F-2 |
| | V2MM_32206 | SM2167-C-12 |
| Hdac9 | V2MM_159316 | SM2202-A-4 |
| Kalrn | V2MM_160069 | SM2130-E-7 |
| | V2MM_84498 | SM2144-F-10 |
| Mapk1 | V2MM_132158 | SM2106-G-2 |
| | V2MM_34173 | SM2396-E-11 |
| Map3k9 | V2MM_70200 | SM2012-G-9 |
| | V2MM_63859 | SM2011-A-5 |
| Mrgbp | V2MM_202249 | SM2487-E-6 |
| | V2MM_105745 | SM2162-H-1 |
| Npm2 | V2MM_93385 | SM2265-E-1 |
| | V2MM_93381 | SM2471-D-1 |
| Pdpk1 | V2MM_78532 | SM2021-G-9 |
| | V2MM_75859 | SM2004-F-9 |
| Ptk2b | V2MM_26156 | SM2434-B-11 |
| | V2MM_21947 | SM2187-E-10 |
| Rcor2 | V2MM_2246 | SM2385-A-12 |
| | V2MM_7624 | SM2604-D-5 |
| Sipa1l2 | V2MM_130034 | SM2106-D-2 |
| | V2MM_130033 | SM2358-E-1 |
| Sirt6 | V2MM_93633 | SM2139-G-10 |
| | V2MM_93636 | SM2451-H-4 |
| Smyd1 | V2MM_74820 | SM2167-E-4 |
| | V2MM_74911 | SM2181-A-12 |
| Sox14 | V2MM_193113 | SM2507-D-4 |
| | V2MM_193113 | SM2298-G-2 |
| S100z | V2MM_150368 | SM2059-C-3 |
| | V2MM_150367 | SM2032-G-11 |
| Trim37 | V2MM_95365 | SM2143-F-9 |
| | V2MM_226566 | SM2464-A-3 |
| Trim66 | V2MM_193395 | SM2269-B-4 |
| | V2MM_93826 | SM2255-H-9 |
| Zcchc4 | V2MM_107407 | SM2612-F-6 |
| | V2MM_202115 | SM2496-B-8 |
| Zfp354b | V2MM_70272 | SM2007-F-3 |
| | V2MM_70504 | SM2026-C-5 |

TABLE 4
Sequences of synthesized shRNAs

| | | SEQ ID |
|---|---|---|
| Gene | Sequence (5′→3′) | NO: |
| Bmi1 | TGCTGTTGACAGTGAGCGCGCAGATGAGGAGAAGAGGAT | 4 |
| | TTAGTGAAGCCACAGATGTAAATCCTCTTCTCCTCATCT | |
| | GCATGCCTACTGCCTCGGA | |
| nmt1 | TGCTGTTGACAGTGAGCGCGCCCATCCTCAGGGACCATA | 5 |
| | TTAGTGAAGCCACAGATGTAATATGGTCCCTGAGGATGG | |
| | GCTTGCCTACTGCCTCGGA | |
| ot1I | TGCTGTTGACAGTGAGCGCGGAGCGATTCGCAAACATGA | 6 |
| | ATAGTGAAGCCACAGATGTATTCATGTTTGCGAATCGCT | |
| | CCTTGCCTACTGCCTCGGA | |
| dac9 | TGCTGTTGACAGTGAGCGCGGACATTTAATTCTGAGATT | 7 |
| | ATAGTGAAGCCACAGATGTATAATCTCAGAATTAAATGT | |
| | CCTTGCCTACTGCCTCGGA | |

Immunoblot analysis. To prepare cell extracts, K-ras NIH 3T3 knockdown cell lines were harvested 7 days following retroviral transduction and puromycin selection (1.5 μg ml⁻¹) and lysed by boiling in 1× SDS sample buffer (Laemmli buffer) for 5 min. Proteins were resolved by 12% SDS-PAGE. Immunoblot analysis was performed using an anti-Fas (sc-716; Santa Cruz) or anti-p21 Ras (ab16795; Abcam) antibody to monitor expression of K-Ras (as a loading control), and an appropriate HRP-conjugated secondary antibody. Proteins were visualized using SuperSignal West Pico Luminol/Enhancer Solution (Pierce).

Bisulphite sequencing. Bisulphite modification was carried out essentially as described (Frommer, M. et al., Proc. Natl. Acad. Sci. USA 89, 1827-1831 (1992)) except that hydroquinone was used at a concentration of 125 mM during bisulphite treatment carried out in the dark and DNA was desalted on Qiaquick columns (Qiagen) after the bisulphite reaction. The regions analyzed were amplified by nested PCR. The first round comprised 24 cycles at 94° C. for 1 min, 48° C. for 1 min 30 s, and 72° C. for 1 min. One-tenth of the product was used as substrate for the second round of PCR comprising 28 cycles at 94° C. for 1 min, 48° C. for 1 min 30 s, 72° C. for 1 min. Primer sequences are provided in Table 5.

TABLE 5
Primer sequences for bisulphite sequencing

| | | | | | |
|---|---|---|---|---|---|
| | | | Forward | | |
| | Position | | (or | | SEQ |
| | (relative to | | reverse | | ID |
| Gene | ISS) | Primer name | primer | Sequence (5′→3′) | NO: |

| | | | | | |
|---|---|---|---|---|---|
| Fas | −30/+260 | FASU2 | forward | GTTGTAGATATGTTGTGGATTTGGGTTG | 8 |
| | | FASR3D2 | reverse | CTAAACAAATCTATAAACCAAAATCCCTCTC | 9 |
| | | FASR3U1 (nested) | forward | GGGTTGTTTTGTTTTTGGTAAGTTTTG | 10 |
| | | FASR3D1 (nested) | reverse | CCAAAATCCCTCTCCAACCATACT | 11 |
| | +260/+623 | FASR4U2 | forward | GGAGAGGGATTTTGGTTTATAGATTTG | 12 |
| | | FASR4D2 | reverse | CCATCCACAATTTAACAACTCAATTCC | 13 |
| | | FASR4D1 (nested) | forward | AAATATCCACCAATTCAACCATCCAC | 14 |
| | | FASR4U1 (nested) | reverse | GTATGGTTGGAGAGGGATTTTGGT | 15 |
| | −2633/−2362 | FAS2.6U | forward | GAAAAGAAGTAGAAATAGAAGTTGAG | 16 |
| | | FAS2.6D | reverse | CTACATCCCAACTATAACTTTACTAC | 17 |
| | −6212/−5970 | FAS6.2U | forward | GTTTGGTTTATAGTTATAGAGTAGAG | 18 |
| | | FAS6.2D | reverse | CACTAAAAAACATCATTACTTACACTAACC | 19 |
| h2 | 1150/+295 | H2K1U1 | forward | GTGAGGTTAGGGGTGGGGGAAGTTTA | 20 |
| | | H2K1D1 | reverse | CTCTTAACTCTCTATATCTACTCCTC | 21 |
| | | H2K1U2 (nested) | forward | GTTTTATTTTTGTTTTTAATTTGGGTTAGG | 22 |
| | | H2K1D2 (nested) | reverse | AAATACCTCAACAAATATAAACCTAAAAA | 23 |
| Lox | +2577/+263 | LOXe3U1 | forward | AGGGAGGGGGTTGTTAGGATTTTG | 24 |
| | | LOXe3U2 (nested) | forward | GTTGTTAGGATTTTGTGATGGTGAGTTG | 25 |
| | | LOXe3D1 | reverse | TAACAACCACCCTCTCTCCTTTCACTC | 26 |
| | | LOXe3D2 (nested) | reverse | CACCCCAAATAAAAAACCCATTCACTTAC | 27 |
| | +4355/+461 | LOXe4U1 | forward | GGAAGTTATTTAGTATTTTTATTGTTTTGTTTATGTG | 28 |
| | | LOXe4U2 (nested) | forward | GTTGTTTTTTTGTTGTGTGGGATATTAGATA | 29 |
| | | LOXe4D1 | reverse | CAACAACTAACTTACTATCACTTTCCTA | 30 |
| | | LOXe4D2 (nested) | reverse | TCCAAATATCAAAAAACCTACCTACCTA | 31 |
| Par4 | +360/+568 | R4F | forward | TTAGGAAAGGTAAGGGGTAGAT | 32 |
| | | R4D | reverse | CAATCATTTACTCCAAATAAAACTCCATC | 33 |
| | −68/+254 | PARTSU | forward | AGTTAGGGATTGTTTTTAGTTTAGG | 34 |
| | | PARTSD | reverse | CACAACTCCCCRAAACTCCCATTC | 35 |
| Plagl | −89/+306 | PLAGLU | forward | ATTTGTTATTTAGTTTGGGTTGGGAT | 36 |
| | | PLAGLD | reverse | CTACATCTCTAACTACAACTAAAACAC | 37 |
| Sfrp1 | −218+516 | SFRPU1 | forward | GAAAGTATTTGTTTAGTTTTTGGTTTTG | 38 |
| | | SFRPD1 | reverse | CAAATTAAACAACACCATTCTTATAACC | 39 |
| | | SFRPU2 (nested) | forward | GTTTTGTTTTTAAGGGGTGTTGAT | 40 |
| | | SFRPD2 (nested) | reverse | TTATAACACAACCTCAAATCCAC | 41 |

Chromatin immunoprecipitation (ChIP) and methylated DNA immunoprecipitation (MeDIP). ChIP assays were performed using extracts prepared 7 days following retroviral transduction and puromycin selection. The following antibodies were used: anti-5-methyl cytosine (ab1884; Abcam), anti-EZH2 (4905; Cell Signaling Technology), anti-CTCF (07-729; Upstate), anti-BMI1 (ab14389; Abcam), anti-DNMT1 (IMG-261A; Imgenex), anti-SIRT6 (ASB-ARP32409; Aviva Systems Biology), anti-TRIM37 (a gift from A. E. Lehesjoki, Folkhalsan Institute of Genetics, Finland), anti-TRIM66 (a gift from R. Losson, IGBMC, France), and anti-NPM2 (a gift from M. M. Matzuk, Baylor College of Medicine, USA). The anti-ZFP354B antibody was raised against a synthetic peptide corresponding to amino acids 126-143 of the murine protein, and affinity purified on a peptide coupled to agarose. The sequences of the primers used for amplifying the MeDIP and ChIP products are provided in Tables 6 and 7. MeDIP and ChIP products were visualized by autoradiography, or analyzed by quantitative real-time PCR using Platinum SYBR Green qPCR SuperMix-UDG with Rox (Invitrogen). Calculation of fold-differences was done as previously described (Pfaff1 M, Nucleic Acids Research Vol 29, No. 9 Page e45, 2001).
Quantitative real time RT-PCR. Total RNA was isolated using TRIZOL (Invitrogen)7 days following retroviral transduction and puromycin selection. Reverse transcription was performed using SuperScript II Reverse Transcriptase (Invitrogen) as per the manufacturer's instructions, followed by quantitative real-time PCR as described above. The sequences of the primers used for quantitative real-time PCR are provided in Table 8.
Soft agar assays. Soft agar assays were performed using the CytoSelect 96-well Cell Transformation Assay (Cell Biolabs) as per the manufacturer's instructions.
Tumor formation assays. 5×10⁶ NIH 3T3, K-ras NIH 3T3, or K-ras NIH 3T3 knockdown cell lines were suspended in 100 μl of serum-free DMEM and injected subcutaneously into the right flank of athymic Balb/c (nu/nu) mice (Taconic). Tumour dimensions were measured every 3 days from the time of appearance of the tumours, and tumour volume was calculated using the formula π/6×(length)×(width)². Animal experiments were performed in accordance with the Institutional Animal Care and Use Committee (IACUC) guidelines.

TABLE 6

Primer sequences Methylated
DNA immunoprecipitation (MeDIP)

| | Position | Forward | | |
|---|---|---|---|---|
| | relative | or reverse | | |
| Gene | to TSS | primer | Sequence (5′→3′_ | SEQ ID |
| Fas | −14 bp | forward | CAGCCCAGAGTAACTCACTTC | SEQ ID NO: 42 |
| | +500 bp | reverse | CATACCCACAGGCAGTCTAGA | SEQ ID NO: 43 |
| | −2.6 kb | forward | GAAGTAGAAACAGAAGCTGAG | SEQ ID NO: 44 |
| | −2.3 kb | reverse | TTGCTACATCCCAACTGTAAC | SEQ ID NO: 45 |
| | −6.2 kb | forward | GGTCTACAGCCACAGAGCAGA | SEQ ID NO 46 |
| | −5.9 kb | reverse | TCTTCTGTCACTAGAGGGCATC | SEQ ID NO: 47 |
| H2−K1 | −50 bp | forward | GCCACTGGTTATAAAGTCCA | SEQ ID NO: 48 |
| | +125 bp | reverse | AAAGCTGTTTCCCTCCCGAC | SEQ ID NO: 49 |
| Lox | +2.6 kb | forward | GCTGCTAGGACCTTGTGATGG | SEQ ID NO: 50 |
| | +2.8 kb | reverse | CACCCCAGATGAGAGGCCCA | SEQ ID NO: 51 |
| | +4.4 kb | forward | GCTGTTTCTTTGTTGTGTGGG | SEQ ID NO: 52 |
| | +4.6 kb | reverse | TCCAGATGTCAGGGGACCTGC | SEQ ID NO: 53 |
| Par4 | −47 bp | forward | CAGGCCGGCGAGTTTGCCGG | SEQ ID NO: 54 |
| | +90 bp | reverse | TGCGGGTGGCCCGGAAGAGC | SEQ ID NO: 55 |
| | +365 bp | forward | GATCGAGAAGAGGAAGCTGC | SEQ ID NO: 56 |
| | +570 bp | reverse | TCTGGGTCGGGGTAACTTCC | SEQ ID NO: 57 |
| PlagII | −36 bp | forward | CGCCCCGAGCCTTGATTTAG | SEQ ID NO: 58 |
| | +184 bp | reverse | ACTCAGGCGTCGCCGTCAGA | SEQ ID NO: 59 |
| Sfrp1 | −68 bp | forward | CTGATTGGCTGCGCGCGGGG | SEQ ID NO: 60 |
| | +182 bp | reverse | GCAGTGCCGGGCCGCGTCCG | SEQ ID NO: 61 |

TABLE 7
Primer sequences for Chromatin immunoprecipitation (ChIP)

| | | | | |
|---|---|---|---|---|
| | | | | Position |
| | Forward or | | | relative to |
| Region | reverse primer | Sequence (5′→3′) | SEQ ID | TSS |

Fas promoter

| | | | | |
|---|---|---|---|---|
| CP/TSS | forward | GCCGCCTGTGCAGTGGTGA | SEQ ID NO: 62 | −234 |
| | reverse | CTGTGTGTGGGCAGCCTGCGGC | SEQ ID NO: 63 | +20 |
| ~1kb | forward | GGCTATAGATCACCTTCATGTA | SEQ ID NO: 64 | −967 |
| | reverse | GCAGTTAACTCAGGGACCAAG | SEQ ID NO: 65 | −722 |
| ~2kb | forward | GCGTTGCCATAGCATGAACT | SEQ ID NO: 66 | −2330 |
| | reverse | GAGTTAGGGGACCATAGTCA | SEQ ID NO: 67 | −2053 |

Gamma satellite DNA

| | | |
|---|---|---|
| forward | TATGGCGAGGAAAACTGAAA | SEQ ID NO: 68 |
| reverse | TTCACGTCCTAAAGTGTGTAT | SEQ ID NO: 69 |

TABLE 8
Primer sequences for Quantitive real−time RT−PCR (qRT−PCR)

| | Forward or reverse | | |
|---|---|---|---|
| Gene | primer | Sequence (5′→3′) | SEQ ID |
| Asf1a | forward | GGCAAAGGTTCAGGTGAACAAT | SEQ ID NO: 70 |
| | reverse | GGATGAGTCCTGCATTCGGAG | SEQ ID NO: 71 |
| Baz2a | forward | CACTCCTCTAGCACCTCACAC | SEQ ID NO: 72 |
| | reverse | GGTGATGGAGGTGTGAGGTG | SEQ ID NO: 73 |
| Bmi1 | forward | TCGGCCAACTTGCAAAAGAA | SEQ ID NO: 74 |
| | reverse | GGGACTGGCAAACAGGAAG | SEQ ID NO: 75 |
| Eid1 | forward | ACCTTGGTCGAGTCGCTTCC | SEQ ID NO: 76 |
| | reverse | AACTCGTCGCCTTCCAGGTC | SEQ ID NO: 77 |
| Ctcf | forward | CACGGGGGAGAAGCCTTATG | SEQ ID NO: 78 |
| | reverse | CGGGTGAATGTTTTCCCACA | SEQ ID NO: 79 |
| Dnmt1 | forward | GAACCATCACCGTGCGAGAC | SEQ ID NO: 80 |
| | reverse | CCAGTGGGCTCATGTCCTTG | SEQ ID NO: 81 |
| Dot1I | forward | CCACCCCATACCAGGACCAT | SEQ ID NO: 82 |
| | reverse | CTGCTGGGCTCATCCTCAGA | SEQ ID NO: 83 |
| Eed | forward | CGAGAGGGGAAGTGTCGACTG | SEQ ID NO: 84 |
| | reverse | GCCTCCCTCCAGGTTCTTGC | SEQ ID NO: 85 |
| Ezh2 | forward | GTAGCATTCAGCGGGGCTCT | SEQ ID NO: 88 |
| | reverse | GGGTTGCATCCACCACAAAA | SEQ ID NO: 87 |
| | forward | GGCTGGATCTGGAGACTGACC | SEQ ID NO: 88 |
| | reverse | CTGCACCTTCAGCACCTCAG | SEQ ID NO: 89 |
| Fas | forward | GATGCACACTCTGCGATGAAG | SEQ ID NO: 90 |
| | reverse | CAGTGTTCACAGCCAGGAGAAT | SEQ ID NO: 91 |
| Hdac9 | forward | GCAGTCCAGGGAGCTAGACG | SEQ ID NO: 92 |
| | reverse | GAGCTGATCATACTGTGCTAAG | SEQ ID NO: 93 |
| H2−K1 | forward | GAGCAGTGGTTCCGAGTGA | SEQ ID NO: 94 |
| | reverse | GGTCTTCGTTCAGGGCGATG | SEQ ID NO: 95 |
| Kalm | forward | CCTGGACCTGTTGCTGATGG | SEQ ID NO: 96 |
| | reverse | CTGGAGCACAGCTGCAGTCA | SEQ ID NO: 97 |
| Lox | forward | CTCATCTGCCTGAAAGCACAC | SEQ ID NO: 98 |
| | reverse | GGGCAAAGAGGTACATCGAAG | SEQ ID NO: 99 |
| Mapk1 | forward | ACAGAGTCCTCCCCGTCTGC | SEQ ID NO: 100 |
| | reverse | GCATGTTTGGGTGCCATCAT | SEQ ID NO: 101 |
| Map,3k9 | forward | AAGAGGATTCCCCCGGACAT | SEQ ID NO: 102 |
| | reverse | ACACATCGCTGCCTTTGGAA | SEQ ID NO: 103 |
| Mrgbp | forward | ACAAGCCTGTCGGGGTGAAT | SEQ ID NO: 104 |
| | reverse | ACTGTGGGGGTCCACATCCT | SEQ ID NO: 105 |
| Npm2 | forward | GGAGCCCTGAAGCCATATTGAG | SEQ ID NO: 106 |
| | reverse | GGCCTCTAAAGGTGCAAGTCT | SEQ ID NO: 107 |
| Par4 | forward | CCCCGAACAGACAGAAGTGGT | SEQ ID NO: 108 |
| | reverse | CTTGCATCAGCCTCACAAGTC | SEQ ID NO: 109 |
| Pdpk1 | forward | GCAACTACGACAATCTCCTG | SEQ ID NO: 110 |
| | reverse | CCTTTCGCTTATCCACTGGA | SEQ ID NO: 111 |
| PlagI1 | forward | GCAGCCACAGTTTCAGTTGC | SEQ ID NO: 112 |
| | reverse | CTCTGGCTCTGGCTCAGGAT | SEQ ID NO: 113 |
| Ptk2b | forward | TCCAGCAGACCTTCCAGCAG | SEQ ID NO: 114 |
| | reverse | CCTTTAGGGCCGATGACCAG | SEQ ID NO: 115 |
| Rcor2 | forward | TGGGGCTATTGCAGAGGTGA | SEQ ID NO: 118 |
| | reverse | CTGCTCAGCCTCCCATTCCT | SEQ ID NO: 117 |
| Sfrp1 | forward | CATCCATGGGGCTACAGTGA | SEQ ID NO: 118 |
| | reverse | TGGCATGGTGAGTTTCAGG | SEQ ID NO: 119 |
| Sipa1I2 | forward | CTCGTCGTGGCCTCAGAGAA | SEQ ID NO: 120 |
| | reverse | TGTGACGGCCTTGGATCACT | SEQ ID NO: 121 |
| Sirt6 | forward | CTTCCCCAGGGACAAACTGG | SEQ ID NO: 122 |
| | reverse | CGGATCTGCAGCGATGTACC | SEQ ID NO: 123 |
| Smyd1 | forward | TGGAGAAGCAGGAGCCAGTG | SEQ ID NO: 124 |
| | reverse | TTGGTAAGCCCTGCCCTCAT | SEQ ID NO: 125 |
| Sox14 | forward | GGTGAAGAGGGAGCGAAGGA | SEQ ID NO: 126 |
| | reverse | CTGTGGGCACCAGAGATTGG | SEQ ID NO: 127 |
| S100z | forward | GCTGGAGATGGCTATGGACAC | SEQ ID NO: 128 |
| | reverse | GCAACCGTCAGAGCTGCCAC | SEQ ID NO: 129 |
| Trim37 | forward | GGAGAAATTGCGGGATGCTC | SEQ ID NO: 130 |
| | reverse | GCCCAACGACAGTTCACCAG | SEQ ID NO: 131 |
| Trim66 | forward | TTTCGTCTGGCCAACAGCAT | SEQ ID NO: 132 |
| | reverse | CTGAAGGATGGGGAGGGATG | SEQ ID NO: 133 |
| Zcchc4 | forward | AGCTTGGAAGGCCCAGTCAG | SEQ ID NO: 134 |
| | reverse | GCCTTGGTGCTCCAACACAC | SEQ ID NO: 135 |
| Zfp354b | forward | GGATGAGTGGAAGAAGCTGG | SEQ ID NO: 136 |
| | reverse | CTCCTTGTTGCAACACGGAG | SEQ ID NO: 137 |

Cell lines and culture conditions. Human HEC1A and HEC1A ras derivative cells (a gift from T. A. Waldman, Georgetown University, USA) were maintained in McCoy's medium supplemented with 10% fetal calf serum (FCS) at 37° C. and 5% CO₂. Murine C3H10T1/2 cells stably transfected with activated human Ha-ras (C3H10T1/2-Ras) and their control counterparts (C3H10T1/2-Neo) (a gift from E. J. Taparowsky, Purdue University, USA) and COS-M6 cells (generously provide by M. Koken, CNRS, France) were maintained in Dulbecco's modified Eagle's medium (DMEM) supplemented with 10% FCS. For 5-aza-2′ deoxycytidine (5-aza) treatment, cells were treated with 10 μM 5-aza for 72 h.
Plasmids. The human ZFP354B open reading frame (accession number BC112111.1) was cloned into the vector PEF6V5b (source) in frame with the C-terminal V5 tag. The PEST sequence deletion derivative (ΔPEST), in which amino acids 80-120 102 were deleted, was derived by PCR using the wild-type expression vector as the substrate, Pfu DNA polymerase (Stratagene) and the following primers: (SEQ ID NO: 138) ZD1 (forward), 5′-GAGAAAGATGCCGGCGGATTTCAGGAGCAGATAAGGAAAAGATTG-3′ and (SEQ ID NO: 139) ZD2 (reverse), 5′-CTCCTGAAATCCGCCGGCATCTTTCTCCACCTCCCAGGGATC-3′. The plasmids pBABE-puro and pBABE-puro-KRASV12 were obtained from Addgene.
Immunoblot analysis. Extract preparation and immunoblot analysis were performed essentially as described in the Methods section accompanying the main text. The PI3K inhibitor LY294002 (LC Laboratories) was added at a concentration of 25 μM for 24 h. Transient cotransfections in COS-M6 cells were performed using Effectene (Qiagen) and, after 24 h, cells were serum-starved for 12 h prior to extract preparation. Antibodies were obtained as follows: anti-ZFP354B antibody (raised against a synthetic peptide corresponding to amino acids 126-143 of the murine protein, and affinity (Kim, J. S., Lee, C., Foxworth, A. & Waldman, T. Cancer Res. 64, 1932-1937 (2004)) purified on a peptide coupled to agarose), anti-Actin (A-5106; Sigma) and anti-Tubulin (T-5368; Sigma).

Quantitative real time RT-PCR. Total RNA was isolated using TRIZOL (Invitrogen)7 days following retroviral transduction and puromycin selection. Reverse transcription was performed using SuperScript II Reverse Transcriptase (Invitrogen) as per the manufacturer's instructions, followed by quantitative real-time PCR as described above. The sequences of the primers used for quantitative real-time PCR are provided in Table 4.

Chromatin immunoprecipitation (ChIP). ChIP assays were performed as described in the Methods section accompanying the main paper, using antibodies anti-DNMT3A (IMG-268A; Imgenex) and anti-DNMT3B (Ab2851; Abcam). The sequences of the primers used for amplifying the ChIP products are provided in Table 4. ChIP products were visualized by autoradiography, or analyzed by quantitative realtime PCR using Platinum SYBR Green qPCR SuperMix-UDG with Rox (Invitrogen). Calculation of fold-differences was done as previously described (Pfaff1, Nucleic Acids Res. 29, e45 (2001)).

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Moreover, this invention is not limited in its application to the details of construction and the arrangement of components set forth in the disclosed description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

We claim:

1. A method for inhibiting gene silencing in a cell comprising reducing expression of one or more Ras epigenetic silencing effectors (RESEs) in the cell.

2. The method of claim 1, wherein the one or more RESEs are encoded by one or more genes of: KALRN, MAPK1, MAP3K1, PDPK1, PTK2B, S100Z, EID1, CTCF, E2F1, RCOR2, SOX14, TRIM66, ZFP354B, BMI1, DNMT1, DOT1L, EED, EZH2, HDAC9, MRGBP, SMYD1, ASF1A, BAZ2A, NPM2, SIRT6, SIPA1L2, TRIM37, and ZCCHC4.

3. The method of claim 1 wherein the one or more RESEs are encoded by one or more genes of: NPM2, TRIM66, ZFP354B, BMI1, DNMT1, SIRT6, TRIM37, EZH2, and CTCF.

4. The method of claim 1, wherein the one or more RESEs are encoded by one or more genes of: BAZ2A, SMYD1, KALRN, S100Z, EID1, TRIM66, MRGBP, TRIM37, and ZCCHC4.

5. The method of claim 1, wherein the one or more RESEs are encoded by one or more genes of: KALRN, S100Z EID1, TRIM66, MRGBP, TRIM37, and ZCCHC4.

6. The method of any one of claims 1-5, wherein the gene is one or more of: FAS, PAR4/MET, LOX, H2-K1, PLAGL1, and SFRP1.

7. The method of any one of claims 1-6, wherein the gene is FAS.

8. The method of any one of claims 1-7, wherein the gene silencing is RAS dependent.

9. The method of any one of claims 1-8, wherein the inhibition of gene silencing comprises decreased DNA methylation.

10. The method of claim 9, wherein the decreased DNA methylation is mediated by DNMT1.

11. The method of any one of claim 1-10, wherein the expression of RESEs is reduced by RNAi against the one or more mRNAs encoding the one or more RESEs.

12. The method of claim 11, wherein the RNAi comprises contacting a cell with a composition comprising a siRNA molecule, shRNA molecule, shRNA-mir molecule, miRNA molecule, or dsRNA molecule.

13. The method of claim 11 or 12, wherein the RNAi comprises contacting a cell with a composition comprising a vector encoding a shRNA or shRNA-mir molecule.

14. A method for inhibiting silencing of a gene in a cell comprising reducing the interaction of one or more Ras epigenetic silencing effectors (RESEs) with a regulatory DNA sequence of the gene in the cell.

15. The method of claim 14, wherein the one or more RESEs are encoded by one or more genes of: NPM2, TRIM66, ZFP354B, BMI1, DNMT1, SIRT6, TRIM37, EZH2, and CTCF.

16. The method of claim 14 or 15, wherein the gene is one or more of: FAS, PAR4/MET, LOX H2-K1, PLAGL1, and SFRP1.

17. The method of claim 14 or 15, wherein the gene is FAS.

18. The method of any one of claims 14-17, wherein the interaction is reduced by RNAi against the one or more mRNAs encoding the one or more RESEs.

19. The method of claim 18, wherein the RNAi comprises contacting a cell with a composition comprising a siRNA molecule, shRNA molecule, shRNA-mir molecule, miRNA molecule, or dsRNA molecule.

20. The method of claim 18 or 19, wherein the RNAi comprises contacting a cell with a composition comprising a vector encoding a shRNA or shRNA-mir molecule.

21. The method of any one of claims 14-20, wherein the regulatory DNA sequence is located about at the transcriptional start site of the gene.

22. The method of any one of claims 14-21, wherein the regulatory DNA sequence is within about 1 kb upstream of the transcriptional start site of the gene.

23. The method of any one of claims 14-22, wherein the regulatory DNA sequence is within about 2 kb upstream of the transcriptional start site of the gene.

24. A method for inhibiting proliferation of a cell comprising reducing expression of one or more Ras epigenetic silencing effectors (RESEs) in the cell.

25. The method of claim 24, wherein the one or more RESEs are encoded by one or more genes of: BAZ2A, SMYD1, KALRN, S100Z, E1D1, TRIM66, MRGBP, TRIM37, and ZCCHC4.

26. The method of claim 24, wherein the one or more RESEs are encoded by one or more genes of: KALRN, S100Z, EID1, TRIM66, MRGBP, TRIM37, and ZCCHC4.

27. The method of any one of claims 24-26, wherein the proliferation of the cell is RAS dependent.

28. The method of any one of claims 24-27, wherein the proliferation of the cell is anchorage independent.

29. The method of any one of claims 24-28, wherein the reducing expression comprises RNAi.

30. The method of claim 29, wherein the RNAi comprises contacting a cell with a composition comprising a siRNA molecule, shRNA molecule, shRNA-mir molecule, miRNA molecule, or dsRNA molecule.

31. The method of claim 29, wherein the RNAi comprises contacting the cell with a composition comprising a vector encoding a shRNA or shRNA-mir molecule.

32. The method of any one of claims 24-31, wherein the cell is in vitro.

33. The method of any one of claims 24-31, wherein the cell is in vivo.

34. The method of claim 33, wherein the cell forms a benign tumor.

35. The method of claim 33, wherein the cell forms a malignant tumor.

36. A method for inhibiting RAS-mediated growth of a tumor comprising reducing expression of one or more Ras epigenetic silencing effectors (RESEs) in the tumor.

37. The method of claim 36, wherein the one or more RESEs are encoded by one or more genes of: BAZ2A, SMYD1, KALRN, 5100Z EID1, TRIM66, MRGBP, TRIM37, and ZCCHC4.

38. The method of claim 36, wherein the one or more RESEs are encoded by one or more genes of: KALRN, S100Z, EID1, TRIM66, MRGBP, TRIM37, and ZCCHC4.

39. The method of any one of claims 36-38, wherein the tumor is benign.

40. The method of any one of claims 36-38, wherein the tumor is malignant.

41. The method of any one of claims 36-40, wherein the tumor is in a subject in need of a treatment that reduces the expression of the one or more RESEs in cells of the tumor.

42. The method of any one of claims 36-41, wherein the reducing expression comprises RNAi.

43. The method of claim 42, wherein the RNAi comprises contacting the tumor with a composition comprising a siRNA molecule, shRNA molecule, shRNA-mir molecule, to miRNA molecule, or dsRNA molecule.

44. The method of claim 41 or 42, wherein the RNAi comprises contacting the tumor with a composition comprising a vector encoding a shRNA or shRNA-mir molecule.

45. The method of claim 43 or 44, wherein the composition is a pharmaceutical composition.

46. A method for screening for regulators of FAS expression comprising:
(i) transducing eukaryotic cells with pools of a plurality of retroviruses, wherein individual retroviruses in the plurality comprise a nucleic acid encoding a product that modulates expression of at least one gene encoded in the genome of the transduced cells;
(ii) isolating FAS positive transduced cells; and
(iii) identifying the transduced nucleic acid.

47. The method of claim 46, wherein the isolating comprises selecting transduced cells containing a genomically integrated portion of the retroviral genome comprising the nucleic acid.

48. The method of claim 46 or 47, wherein the genomically integrated portion of the retroviral genome further comprises a sequence encoding a product that confers resistance to a compound.

49. The method of claim 48, wherein the product that confers resistance to a compound is N-puromycin acetyltransferase.

50. The method of any one of claims 47-49, wherein the selecting comprises contacting the transduced cells with a compound that is inactivated by the product that confers resistance.

51. The method of any one of claims 48-50, wherein the compound is Puromycin.

52. The method of any one of claims 46-51, wherein the isolating comprises immunoaffinity purification.

53. The method of claim 52, wherein the immunoaffinity purification comprises contacting the transduced cells with an antibody or antigen binding fragment thereof that binds to FAS.

54. The method of any one of claims 46-53, wherein the identifying comprises isolating the genomically integrated portion of the retroviral genome comprising the nucleic acid.

55. The method of claim 54, wherein the isolated nucleic acid is sequenced.

56. The method of any one of claims 46-55, wherein the product that modulates expression is an shRNA or shRNA-mir.

57. The method of claim 56, wherein the shRNA or shRNA-mir is directed against the at least one gene encoded in the genome of the transduced cells.

58. The method of any one of claims 46-57, wherein the plurality of retroviruses comprise sequence complementary to a portion of the mRNA sequence of each of substantially all known protein coding genes of the transduced cell's genome.

59. A method for identifying compounds or compositions that inhibit RAS-mediated tumor growth comprising contacting a cell with a compound or composition and assaying for decreased expression of one or more Ras epigenetic silencing effectors (RESEs).

60. The method of claim 59, wherein the one or more RESEs are encoded by one or more genes of: KALRN, MAPK1, MAP3K9, PDPK1, PTK2B, S100Z, EID1, CTCF, E2F1, RCOR2, SOX14, TRIM66, ZFP354B, BMI1, DNMT1, DOT1L, EED, EZH2, HDAC9, MRGBP, SMYD1, ASF1A, BAZ2A, NPM2, SIRT6, SIPA1L2, TRIM37, and ZCCHC4.

61. The method of claim 59 or 60, further comprising assaying for altered expression of one or more of: FAS, PAR4/MET, LOX, H2-K1, PLAGL1, and SFRP1 in the cell.

62. The method of any one of claims 59-61, further comprising assaying for altered DNA methylation at regulatory DNA sequences of one or more of: FAS, PAR4/MET, LOX H2-K1, PLAGL1, and SFRP1 in the cell.

63. The method of any one of claims 59-62, further comprising assaying for altered interaction of DNMT1 with regulatory DNA sequences of one or more of: FAS, PAR4/MET, LOX, H2-K1, PLAGL1, and SFRP1 in the cell.
